SEQUENCE LISTING (5 sequences)

SEQ ID NO: 1 - DNA, 1503 bp, Human immunodeficiency virus type 1
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaagaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcagatcactctttggcaacgacccctcgtcacaataa

SEQ ID NO: 2 - protein, 582 aa, Mumps virus
MEPSKLFIMSDNATFAPGPVVNAAGKKTFRTCFRILVLSVQAVTLILVIVTLGELIRMINDQGLSNQLSSITDKIRESAAMIASAVGVMNQVIHGVTVSLPLQIEGNQNQLLSTLATICTNRNQVSNCSTNIPLVNDLRFINGINKFIIEDYATHDFSIGHPLNMPSFIPTATSPNGCTRIPSFSLGKTHWCYTHNVINANCKDHTSSNQYVSMGILVQTASGYPMFKTLKIQYLSDGLNRKSCSIATVPDGCAMYCYVSTQLETDDYAGSSPPTQKLTLLFYNDTIKERTISPSGLEGNWATLVPGVGSGIYFENKLIFPAYGGVLPNSTLGVKSAREFFRPVNPYNPCSGPPQELDQRALRSYFPSYFSSRRVQSAFLVCAWNQILVTNCELVVPSNNQTLMGAEGRVLLINNRLLYYQRSTSWWPYELLYEISFTFTNSGQSSVNMSWIPIYSFTRPGLGKCSGENICPTVCVSGVYLDPWPLTPYSHQSGINRNFYFTGALLNSSTTRVNPTLYVSALNNLKVLAPYGTQGLFASYTTTTCFQDTGDASVYCVYIMELASNIVGEFQILPVLARLTIT

SEQ ID NO: 3 - protein, 159 aa, mumps virus
MGILVQTASGYPMFKTLKIQYLSDGLNRKSCSIATVPDGCAMYCYVSTQLETDDYAGSSPPTQKLTLLFYNDTIKERTISPSGLEGNWATLVPGVGSGIYFENKLIFPAYGGVLPNSTLGVKSAREFFRPVNPYNPCSGPPQELDQRALRSYFPSYFSS

SEQ ID NO: 4 - protein, 9 aa, mumps virus
GPPQELDQR

SEQ ID NO: 5 - protein, 538 aa, mumps virus
MKAYPVICLGFAIFSSSICVNINILQQIGYIKQQVRQLSYYSQSSSSYVVVKLLPNIQPTDNSCEFKSVTQYNKTLSNLLLPIAENINNIASPSPGSRRHKRFAGIAIGIAALGVATAAQVTAAVSLVQAQTNARAIAAMKNSIQATNRAVFEVKEGTQQLAIAVQAIQDHINTIMNTQLNNMSCQILDNQLATSLGLYLTELTTVFQPQLINPALSPISIQALRSLLGSMTPAVVQATLSTSISAAEILSAGLMEGQIVSVLLDEMQMIVKINVPTIVTQSNALVIDFYSISSFINNQESIIQLPDRILEIGNEQWRYPAKNCKSTRHHIFCQYNEAERLSLETKLCLAGNISACVFSPIAGSYMRRFVALDGTIVANCRSLTCLCKSPSYPIYQPDHHAVTTIDLTSCQTLSLDGLDFSIVSLSNITYAENLTISLSQTINTQPIDISTELSKVNASLQNAVKYIKESNHQLQSVSVSSKIGAIIVAALVLSILSIIISLLFCCWAYIATKEIRRINFKTNHINTISSSVDDLIRY

(19) United States
(12) Patent Application Publication
(10) Pub. No.: US 2009/0092635 A1
(43) Pub. Date: Apr. 9, 2009

(54) HETEROLOGOUS PRIME-BOOST IMMUNIZATION REGIMEN

(75) Inventors: David Kirkwood Clarke, Chester, NY (US); Farooq Nasar, Albany, NY (US); Rong Xu, Bardonia, NY (US); Shakuntala Devi Megati, New City, NY (US); Michael Albin Egan, Washingtonville, NY (US); Amara Gbellu Luckay, Old Bridge, NJ (US)

Correspondence Address: WYETH;PATENT LAW GROUP, 5 GIRALDA FARMS, MADISON, NJ, 07940, US

(73) Assignee: WYETH, Madison, NJ (US)

(21) Appl. No.: 12/192,257

(22) Filed: Aug. 15, 2008

Related U.S. Application Data
(60) Provisional application No. 60/965,285, filed on Aug. 17, 2007.

(51) Int. Cl. A61K 39/12; A61K 39/165; A61P 37/04; A61P 31/12

(52) U.S. Cl. 424/199.1

(57) ABSTRACT

The present invention is directed to a method for generating an antigen-specific immune response in a subject in general and in particular to administering a priming dose of an immunogenic composition of a recombinant mumps virus (rMuV) that encodes an antigen followed by administering a boosting dose of recombinant vesicular stomatitis virus (rVSV) encoding an antigen.

### CROSS REFERENCE TO RELATED APPLICATIONS

This application claims priority from U.S. Provisional Application No. 60/965,285, filed Aug. 17, 2007, which is incorporated by reference in its entirety.

### FIELD OF THE INVENTION

The present invention is related to the fields of virology, infectious disease and immunology. More particularly, the present invention is directed to a method for generating an antigen-specific immune response by the sequential administration of a priming dose of an immunogenic composition comprising a recombinant mumps virus that encodes an antigen, followed by administration of a boosting dose of an immunogenic composition comprising a recombinant vesicular stomatitis virus composition that encodes an antigen.

### BACKGROUND OF THE INVENTION

The ability to recover infectious virus from genomic cDNA has enabled the development of non-segmented negative strand RNA viruses as expression vectors for proteins and candidate vectors for immunization. Vesicular stomatitis virus (VSV), which predominantly infects insects and livestock in nature, is one of the most extensively studied of these viruses, and recombinant forms of VSV (rVSV) have been tested in pre-clinical studies as potential vectors for immunization to combat a wide range of human diseases including HIV-1 induced AIDS. In one of these studies, non-human primates (NHPs) immunized with rVSV immunogenic composition vectors expressing SIV Gag and HIV Env proteins were protected from disease following challenge with a pathogenic simian immunodeficiency virus (SIV), HIV-1 recombinant (SHIV) (Rose et al. Cell 106: 539-549). Although these prototypic rVSV immunogenic compositions elicited robust SIV/HIV-1 specific immune responses in NHPs and had good protective efficacy in the SHIV challenge model, they were found to be insufficiently attenuated for human trials when tested in a stringent NHP neurovirulence (NV) model (Johnson et al. Virology 360: 36-49). To overcome this problem, a highly attenuated rVSV vector was developed by combination of specific N gene translocations and G gene truncations, with the HIV-1 gag gene added in the first position of the genome, the N gene in position 4 and expressing a G protein with a single amino acid in the cytoplasmic tail (rVSVN4CT1gag1). See published internation patent application WO 2005/098009.

Mumps virus (MuV), the causative agent of mumps in humans, is a non-segmented negative strand RNA virus in the family paramyxoviridae. The incidence of mumps has been greatly reduced in the developed world by the introduction of live attenuated MuV immunogenic composition strains over the past 30-35 yrs. The most commonly used MuV immunogenic composition in the USA and Western Europe is the Jeryl Lynn strain, which has demonstrated excellent efficacy and an outstanding safety record, for the approximately 100 million doses administered to the pediatric population. A system for the recovery of the Jeryl Lynn strain of MuV from genomic cDNA has been described previously. Clarke D. K. et al., 2000, J. Virol., 74:4831-4838. This methodology has enabled targeted alteration of the MuV genome to study virus-associated neurovirulence, neuroattenuation and the possibility of developing MuV as a vector for immunization.

### SUMMARY OF THE INVENTION

In one aspect, the invention provides methods for generating an antigen specific immune response in a subject, the method comprising in sequential order the steps of: (a) administering to the subject at least one dose of a first immunogenic composition comprising a recombinant mumps virus (rMuV) comprising a nucleic acid sequence encoding a heterlogous antigen under the control of a regulatory sequence that directs expression of the antigen by the recombinant rMuV; followed by (b) administering to the subject at least one dose of a second immunogenic composition comprising a recombinant vesicular stomatitis virus (rVSV) comprising a nucleic acid sequence encoding a heterologous antigen under the control of a regulatory sequence that directs expression of the antigen by the recombinant rVSV, wherein the antigen expressed in step (a) may be the same as or different from the antigen expressed in step (b).

In some embodiments, the invention provides methods for generating an antigen specific immune response in a subject by administering immunogenic compositions in sequential order, wherein the first immunogenic composition comprises an rMuV having an alteration in an rMuV protein. Alteration of the epitope results in an rMuV that is no longer neutralized by a pre-existing neutralizing antibody response to MuV that may be present in the subject. In some embodiments the rMuV comprises an altered epitope in the HN protein. In some embodiments the rMuV comprises an altered epitope in the F protein.

In some embodiments, the invention provides methods for generating an antigen specific immune response as described herein wherein at least one of said immunogenic compositions is administered in a pharmaceutically acceptable diluent.

In some embodiments, the invention provides methods for generating an antigen specific immune response as described herein wherein the first immunogenic composition is administered to the subject at least two times prior to administering to the subject the second immunogenic composition.

In some embodiments, the invention provides methods for generating an antigen specific immune response as described herein wherein the subject is a human.

In some embodiments, the invention provides methods for generating an antigen specific immune response as described herein, wherein at least one of the rMuV and rVSV is attenuated.

In some embodiments the invention provides methods for generating an antigen specific immune response as described herein, wherein the rVSV is attenuated and expresses a G protein having a truncated cytoplasmic tail (CT) domain. In some embodiments, the attenuated rVSV expresses a G protein having the cytoplasmic domain truncated to one amino acid (CT1).

In some embodiments the invention provides methods for generating an antigen specific immune response as described herein, wherein the attenuated rVSV expresses a G protein having a cytoplasmic domain truncated to nine amino acids (CT9). In some embodiments the invention provides methods for generating an antigen-specific immune response as described herein wherein the rVSV is attenuated by the translocation of the N gene to a different, distal position in the genome.

In some embodiments, the invention provides methods for generating an antigen specific immune response as described herein, wherein the rVSV is attenuated and is propagation defective. In some embodiments, the propagation incompetent rVSV lacks a VSV G protein, VSV)G. In some embodiments, the propagation defective rVSV expresses a G protein having a truncated extracellular domain (rVSV-Gstem).

In some embodiments, the invention provides methods for generating an immune response as described herein wherein the rMuV is attenuated. In some embodiments, the attenuated rMuV is derived from the Jeryl Lynn strain.

In some embodiments, the invention provides methods for generating an immune response as described herein which further comprise administering to the subject one or more adjuvants.

In some embodiments, the invention provides methods for generating an immune response as described herein wherein the antigen is a protein, wherein the protein is derived from a source selected from the group consisting of a bacterium, virus, fungus, parasite, a cancer cell, a tumor cell, an allergen and a self-molecule.

In some embodiments, the invention provides methods for generating an immune response as described herein wherein the antigen is selected from the group consisting of: an HIV antigen, an HTLV antigen, an SIV antigen, an RSV antigen, a PIV antigen, an HSV antigen, a CMV antigen, an Epstein-Barr virus antigen, a Varicelia-Zoster virus antigen, a mumps virus antigen, a measles virus antigen, an influenza virus antigen, a poliovirus antigen, a rhinovirus antigen, a hepatitis A virus antigen, a hepatitis B virus antigen, a hepatitis C virus antigen, a Norwalk virus antigen, a togavirus antigen, an alphavirus antigen, a rubella virus antigen, a rabies virus antigen, a Marburg virus antigen, an Ebola virus Plasmodium knowlesi antigen, a Streptococcus pneumoniae antigen, Streptococcus pyogenes antigen, a Helicobacter pylon antigen, a Streptococcus agalactiae antigen, a Neisseria meningitidis antigen, a Neisseria gonorrheae antigen, a Corynebacteria diphtheriae antigen, a Clostridium tetani antigen, a Bordetella pertussis antigen, a Haemophilus antigen, a Chlamydia antigen, a Escherichia coli antigen, a cytokine, a T-helper epitope, and a CTL epitope.

In some embodiments, the invention provides methods for generating an immune response as described herein wherein the antigen comprises an HIV antigen. In some embodiments, the invention provides methods for generating an immune response as described herein wherein the HIV antigen comprises a protein encoded by a gene selected from the group consisting of gag, env, pol, vif, nef, tat, vpr, rev or vpu. In some embodiments, the invention provides methods for generating an immune response as described herein wherein the HIV antigen is a gag protein. In some embodiments, the invention provides methods for generating an immune response as described herein wherein the nucleic acid that encodes the gag protein comprises SEQ ID NO.1.

In some embodiments, the invention provides methods for generating an immune response as described herein wherein the immune response comprises an increase in cell mediated immunity greater than that achieved by administering the first or second immunogenic compositions alone.

In some embodiments, the invention provides methods for generating an immune response as described herein wherein the immune response comprises an increase in antibody response to the antigen greater than that achieved by administering the first or second compositions alone.

In some embodiments, the invention provides methods for generating an immune response as described herein that can further comprise administering to the subject another antigen.

In some embodiments, the invention provides methods for generating an immune response as described herein wherein at least one of the first and second immunogenic compositions is administered by a route selected from the group consisting of: intravenous, intradermal, subcutaneous, intramuscular, intraperitoneal, oral, rectal, intranasal, buccal, and vaginal.

In some embodiments, the invention provides methods for generating an immune response as described herein wherein the first immunogenic composition is administered using an intramuscular or subcutaneous route.

In some embodiments, the invention provides methods for generating an immune response as described herein wherein the second immunogenic composition is administered using an intramuscular or subcutaneous route.

In some embodiments, the invention provides methods for generating an immune response as described herein wherein the second immunogenic composition is administered using an intramuscular or subcutaneous route.

In some embodiments, the invention provides methods for generating an immune response as described herein wherein the second immunogenic composition is administered at least once between about 4 weeks and about 10 weeks after the first immunogenic composition is administered at least once.

In some embodiments, the invention provides methods for generating an immune response as described herein wherein the second immunogenic composition is administered at least once between about 7 weeks and about 9 weeks after the first immunogenic composition is administered at least once.

In some embodiments, the invention provides methods for generating an immune response as described herein wherein the second immunogenic composition is administered at least once at about 8 weeks after the first immunogenic composition is administered at least once.

In another aspect, the invention provides an immunogenic composition for generating an antigen-specific immune response in a subject, the immunogenic composition comprising: a first immunogenic composition comprising a recombinant mumps virus (rMuV) comprising a nucleic acid sequence encoding a heterologous antigen under the control of a regulatory sequence that directs expression of the antigen by the recombinant rMuV; and a second immunogenic composition to be administered after the first immunogenic composition, said second composition comprising at least one recombinant vesicular stomatitis virus (rVSV) comprising a nucleic acid sequence encoding a heterologous antigen under the control of a regulatory sequence that directs expression of the antigen by the recombinant VSV, wherein the antigen expressed in the first composition may the same as or different from the antigen expressed in the second composition.

In some embodiments, the invention provides an immunogenic composition for generating an antigen specific immune response in a subject as described herein wherein the first immunogenic composition comprises an rMuV having an alteration in an rMuV protein. Alteration of the epitope results in an rMuV that is no longer neutralized by a pre-existing neutralizing antibody response to MuV that may be present in the subject. In some embodiments the rMuV comprises an altered epitope in the HN protein. In some embodiments the rMuV comprises an altered epitope in the F protein.

In some embodiments, the immunogenic composition of the invention for generating an immune response as described herein wherein the subject is a human.

In another aspect, the invention provides a kit for generating an antigen specific response in a subject, the kit comprising the first immunogenic composition comprising a recombinant mumps virus (rMuV) comprising a nucleic acid sequence encoding a heterologous antigen under the control of a regulatory sequence that directs expression of the antigen by the recombinant rMuV, and a second immunogenic composition to be administered after the first immunogenic composition comprising a recombinant vesicular stomatitis virus (rVSV) comprising a nucleic acid sequence encoding a heterologous antigen under the control of a regulatory sequence that directs expression of the antigen by the recombinant rVSV, wherein the antigen expressed in the first composition may the same as or different from the antigen expressed in the second composition. In some embodiments, the invention provides kits for generating an immune response in a subject as described herein wherein at least one of the rMuV and rVSV is propagation competent.

In some embodiments, the invention provides for kits for generating an immune response in a subject wherein the first immunogenic composition comprises an alteration in one or more rMuV proteins. Alteration of the epitope results in an rMuV that is no longer neutralized by a pre-existing neutralizing antibody response to MuV that may be present in the subject. In some embodiments the rMuV comprises an altered epitope in the HN protein. In some embodiments the rMuV comprises an altered epitope in the F protein.

In some embodiments, the invention provides kits for generating an immune response in a subject as described herein wherein at least one of the rMuV and rVSV is propagation competent or wherein at least one of the rMuV and rVSV is propagation incompetent. In some embodiments, the invention provides kits for generating an immune response in a subject as described herein wherein the rVSV is propagation competent. In some embodiments, the invention provides kits for generating an immune response in a subject as described herein wherein the rVSV is propagation incompetent.

In some embodiments, the invention provides a kit for generating an immune response in a subject as described herein and further comprising an adjuvant.

In some embodiments, the invention provides for the use of an immunogenic composition in the manufacture of a medicament for priming an immune response to an antigen wherein the immunogenic composition comprises a rMuV comprising a nucleic acid encoding a heterologous antigen under the control of a regulatory sequence that directs expression of the antigen by the recombinant rMuV, wherein the priming is followed by the use of a second immunogenic composition used in the manufacture of a medicament for boosting an immune response to the antigen, wherein the second immunogenic composition comprises an rVSV comprising a nucleic acid encoding the antigen under the control of a regulatory sequence that directs expression of the antigen by the recombinant rVSV.

In some embodiments, the invention provides for the use of an immunogenic composition in the manufacture of a medicament for boosting an immune response to an antigen, wherein the immunogenic composition comprises an rVSV comprising a nucleic acid encoding a heterologous antigen under the control of a regulatory sequence that directs expression of the antigen by the recombinant rVSV and wherein the boosting is preceded by the use of a first immunogenic composition for priming an immune response to the antigen, wherein the first immunogenic composition comprises an rMuV comprising a nucleic acid encoding the antigen under the control of a regulatory sequence that directs expression of the antigen by the recombinant rMuV.

### BRIEF DESCRIPTION OF THE DRAWINGS

FIG. 1 is a graphical representation showing genetic organization of the MuV genome and insertion site for insertion of HIV-1 p55 gag. Le and Tr denote non-coding viral leader and trailer sequence, respectively. N, P, M, F, SH, HN and L denote transcription units (TUs) encoding the respective MuV proteins. An Ascl site was generated in the M gene UTR by PCR mutagenesis. The HIV-1 p55 gag gene with flanking M gene transcription stop and F gene transcription start signals was cloned into the Ascl site.

FIG. 2 is a graphical representation of a Western blot analysis of HIV-1 gag protein expressed in Vero cells by rMuVgag and rVSVN4CT1gag1.

FIG. 3 is a graphical representation showing bar graphs of HIV-1 gag-specific cellular immune responses. Panel A. HIV-1 gag-specific IFN-6 ELISPOT responses in immunized animals. Panel B. HIV-1 gag-specific IL-2 ELISPOT responses in immunized animals.

FIG. 4 is a graphical representation showing bar graphs and pie charts showing ICS in immunized animals at wk 25. Panel A. Average percentage of CD8+ cells expressing IFN-δ, IL-2 and TNF-α, with standard error (I). Panel B. Average percentage of CD4+ cells expressing IFN-δ, IL-2 and TNF-α, with standard error (I).

FIG. 5 is a graphical representation showing average MuV-specific IgG (Panel A) and neutralization titers (Panel B) for the different immunization regimens.

FIG. 6 is a graphical representation of MuV neutralization titers. Serum samples from rhesus macaques were taken at intervals following inoculation with rMuV, and rMuVgag, and tested in a standard neutralization assay for MuV.

FIG. 7 is a graphical representation of MuV-Specific IgG Responses. Serum samples from rhesus macaques were taken at intervals following inoculation with rMuV and assayed for the presence of MuV-specific IgG by ELISA.

FIG. 8 is a tabular representation of individual data for human MuV specific IgG titers and neutralization responses that result from previous exposure to MuV.

FIG. 9 is a graphical representation showing HIV-1 Gag-Specific ELISPOT Responses. Peripheral blood lymphocytes (PBLs) were assayed for gag-specific γ-IFN ELISPOT production at intervals following inoculation of macaques with rMuVgag and rVSVN4CT1gag1. No gag-specific ELISPOT response was detected after priming with rMuVgag (data not shown). At wk 2 post boost with rVSVN4CT1gag1 an average of 150 gag-specific ELISPOTS were detected, declining to an average of ˜30 ELISPOTS at wk 3 post boost.

### BRIEF DESCRIPTION OF THE SEQUENCES

| SEQUENCE | DESCRIPTION OF SEQUENCE |
|---|---|
| SEQ ID NO. 1 | HIV-1 full length gag nucleic acid sequence |
| SEQ ID NO. 2 | Full length MuV HN protein |
| SEQ ID NO. 3 | Amino acids 213-372 of the HN3 domain of the MuV |
| | HN protein |
| SEQ ID NO. 4 | HN3 domain amino acids 352-360 |
| SEQ ID NO. 5 | Full length MuV F protein |

### DETAILED DESCRIPTION

The invention provides a method of inducing an antigen-specific immune response in a mammalian subject or vertebrate, particularly human, subject by using in combination certain components of immunogenic compositions and optimizing the components to produce synergistic results. Generally, the method involves administering to the subject an effective amount of a composition that includes a recombinant attenuated mumps virus (rMuV) comprising a DNA sequence encoding a heterologous antigen under the control of regulatory sequences directing expression thereof in a mammalian or vertebrate cell by the rMuV. The method also includes a step of administering to the subject an effective amount of a composition comprising a recombinant attenuated vesicular stomatitis virus in particular the construct designated rVSVN4CT1. This rVSVN4CT1 construct comprises a nucleic acid sequence encoding a heterologous antigen under the control of regulatory sequences directing expression thereof in the mammalian or vertebrate cell by the rVSVN4CT1. The heterologous antigen expressed by rMuV and rVSVN4CT1 may be the same or different antigens.

Currently, there is no proven method of inducing broadly neutralizing antibodies using HIV-1 immunogenic compositions. However, it has been postulated that very robust immunogenic composition-induced cellular immune responses directed against one or more HIV-1 proteins may be sufficient to protect humans from AIDS in the absence of broadly neutralizing antibodies. One approach to maximizing immunogenic composition-induced peak cellular immune responses to HIV-1 proteins is administration of heterologous vectors for immunization in prime-boost regimens.

The first of these two immunogenic compositions to be administered in order is referred to as the priming composition. The second of these two immunogenic compositions to be administered in order is referred to as the boosting composition. In particular the rMuV priming composition is administered as the priming composition and the rVSV composition, for example the rVSVN4CT1 composition, is administered as the boosting composition. In some embodiments the priming composition is administered to the subject at least once or multiple times prior to administration of the boosting composition. Thereafter, the boosting composition is subsequently administered to the subject at least once or multiple times after at least one administration of the priming composition. Further the invention contemplates multiple administrations of the priming composition followed by multiple administrations of the boosting composition. The method further contemplates administering an effective amount of an adjuvant and/or a cytokine as a step in the method.

It has been found that administration of a priming dose of rMuV followed by a boosting dose of rVSV, both encoding the same antigen, induces in a mammalian subject an immune response including an increase in CD8+ T cell response to the antigen greater than that achieved by administering either the first or second immunogenic composition alone. Thus, the immune response induced by this novel method is a synergistic increase in cellular responses to the antigen. Immunization of animals with a single dose of rMuV expressing the HIV gag protein elicited a measurable MuV-specific IgG response as well, which was significantly boosted following a second rMuVgag inoculation. Relatively similar results were obtained in MuV neutralization assays, where neutralization titers were very low following a single dose of rMuVgag, but increased significantly after a second rMuVgag inoculation.

While it is contemplated that the mammalian subject is a primate, preferably a human, the invention is not limited by the identification of the mammalian subject. The components of this method are described in detail below and with reference to the cited documents that are incorporated by reference to provide detail known to one of skill in the art.

In one embodiment the present invention relates to a method of administering an immunogenic composition comprising a recombinant attenuated mumps virus expressing HIV-1 gag, rMuVgag, followed by administration with a highly attenuated vesicular stomatitis virus also expressing HIV-1 gag, rVSVN4CT1gag1, in a heterologous prime-boost combination. The HIV-1 gag gene may have the sequence set forth in SEQ ID. NO: 1. In one embodiment the mumps virus has been altered in the HN protein to either diminish or eliminate binding of pre-existing neutralizing antibodies that may be present in humans due to natural infection with MuV or due to precious vaccination with rMuV or MuV. MuV-neutralizing epitopes in the HN or F proteins could react with pre-existing neutralizing antibodies that might be present in a subject, thus preventing replication and gene expression of rMuV This combination and order of administration generates an enhanced immune response when compared to separate administrations or compositions dosed in a different order. The invention addresses a need in the art for an administration regimen that will produce robust cellular responses directed towards one or more antigens. Currently, there is no proven method of inducing broadly neutralizing antibodies using HIV-1 immunogenic composition thus, an approach to increasing immunogenic composition induced peak cellular immune responses to HIV-1 proteins by the administration of a heterologous prime-boost combination is described.

In previous efforts to enhance the efficacy of immunogenic compositions, a variety of immunogenic compositions and methods have been reported using protein compositions, plasmid-based compositions, and recombinant virus constructs encoding antigens as immunogenic compositions.

An often-used DNA prime/live vector boost regimen involves vaccinia viruses for the boost. Examples in the recent literature include such immunization for human immunodeficiency virus (HIV) (Hanke T. et al, 2002 Vaccine, 20:1995-8, Amara R. R. et al, 2002 Vaccine, 20:1949-55, Wee E. G. et al, 2002 J. Gen. Virol., 83:75-80, Amara R. R. et al, 2001 Science, 292:69-74). Prime-boost immunizations with DNA and modified vaccinia virus vectors expressing antigens such as herpes simplex virus-2 glycoprotein D, Leishmania infantum P36/LACK antigen, Plasmodium falciparum TRAP antigen, HIV/SIV antigens, murine tuberculosis antigens, and influenza antigens, have been reported to elicit specific antibody and cytokine responses (See, e.g., Meseda C. A. et al., 2002 J. Infect. Dis., 186:1065-73, Amara R. R. et al, 2002 J. Virol., 76:7625-31; Gonzalo R. M. et al, 2002 Vaccine, 20:1226-31; Schneider J. et al, 2001 Vaccine, 19:4595-602; Hel Z. et al, 2001 J. Immunol., 167:7180-91; McShane H. et al., 2001 Infect. & Immunol., 69:681-6, and Degano P. et al 1999 Vaccine, 18:623-32).

Plasmid prime-adenovirus boost genetic immunization regimens have been reported to induce alpha-fetoprotein-specific tumor immunity and to protect swine from classical swine fever (See, e.g., Meng W. S. 2001 Cancer Res., 61:8782-6; Hammond, J. M. et al., 2001 Vet. Microbiol., 80:101-19; and U.S. Pat. No. 6,210,663). Other DNA plasmid prime-virus boost regimens have been reported. See, e.g., Matano T. et al, 2001). DNA priming with recombinant poxvirus boosting has been reported for HIV-1 treatment (See, e.g., Kent, S. J. et al, 1998; Robinson, H. L. et al, 1999 and Tartaglia, J. et al, 1998 AIDS).

While a number of DNA prime/viral boost regimens are being evaluated, currently described immunization regimens may have certain disadvantages. For example, some of these above-noted viruses cause disease symptoms in subjects; others may result in recombination in vivo. Still other viruses are difficult to manufacture and/or have a limited ability to accept foreign genes. Still other viruses have disadvantages caused by significant pre-existing vector immunity in man, and other safety concerns.

Previous prime boost regimens have been used to test different combinations of heterologous viral vectors and plasmid DNA vectors. In general, the resulting cellular immune responses elicited were higher than those induced in homologous prime boost strategies (Egan et al. 2000 J. Virol., 74:7485-95, Rose et al. 2001 Cell, 106: 539-49), although any associated enhancement of protection in challenge models has not always been clear (Amara et al. 2002, J. Virol., 76:7625-31). Methods have been employed to enhance the efficacy of immunogenic compositions in eliciting an immune response specifically generated by HIV-1 proteins by administering attenuated recombinant viruses expressing one or more HIV-1 proteins. These attenuated recombinant viruses are administered in a prime-boost regimen where one recombinant virus encoding an antigen is administered prior to a second recombinant virus encoding the same antigen.

The priming immunogenic composition used in this invention comprises the recombinant mumps virus (rMuV). The Jeryl Lynn strain of MuV used in this study has the ability to infect and disseminate throughout the human host and has an excellent safety profile in young infants. The approximately 15 kilobase (kb) genome of MuV can also be readily manipulated to robustly express one or more foreign proteins under control of the single 3′ transcription promoter. In addition, MuV is related to measles virus (MV), which has also been modified to express HIV-1 proteins and has shown potential as an HIV-1 vector for immunization (Lorin et al., Therapie 2005 May-June:60(3):227-233, Lorin et al., Vaccine 2005 Aug 22;23(36):4463-72, Lorin et al., 2004 J. Virol 2004 Jan; 78(1):146-157.

The mumps virus was originally classified with influenza viruses in the Myxovirus family but has since been re-assigned to the Paramyxoviridae family, subfamily Paramyxovirinae, genus Rubulavirus, based on nucleocapsid morphology, genome organization and biological properties of the proteins. Other examples of the Rubulavirus genus include simian virus 5 (SV5), human parainfluenza virus type 2 and type 4 and Newcastle disease virus (Lamb and Kolakofsky, 1996 Paramyxoviridae. The viruses and their replication. In “Virology” (B N Fields, D M Knipe, and P M Howley, Eds.) 3^{rd} edition, Voll, pp 1177-1204. Raven Press, New York). Like all viruses of the Paramyxoviridae, mumps virus is pleomorphic in shape, comprising a host cell derived lipid membrane surrounding a ribonucleoprotein core; this nucleocapsid core forms a helical structure composed of a 15,384 nucleotide nonsegmented negative sense RNA genome closely associated with virus nucleocapsid protein (NP). The genetic organization of the MuV genome has been determined to be 3′-NP-P-M-F-SH-HN-L-5′ (Elango et al., 1998). Each gene encodes a single protein except for the P cistron, from which three unique mRNAs are transcribed; one is a faithful copy of the P gene, encoding the V protein, the two other mRNAs contain two and four non-templated G residues inserted during transcription by a RNA editing mechanism, and encode the P and I proteins respectively (Paterson and Lamb, 1990 J. Virol., 64:4137-4145). The P and L proteins in association with nucleocapsid form the functional RNA polymerase complex of mumps virus. The F and HN proteins are integral membrane proteins that project from the surface of the virion, and are involved in virus attachment and entry of cells. The small hydrophobic protein (SH) and matrix (M) protein are also membrane associated (Takeuchi et al, 1996 Virol., 225:156-162, and Lamb and Kolakofsky, 1996).

The replicative cycle of mumps virus initiates upon fusion of virus envelope with host cell plasma membrane and subsequent release of virus nucleocapsid into the cell cytoplasm. Primary transcription then ensues, resulting in the production of all virus proteins; a switch to replication of the virus genome occurs later, followed by assembly of virus components to form new virus particles that bud from the host cell plasma membrane. Only the intact nucleocapsid structure can act as the template for RNA transcription, replication and subsequent virus amplification. The naked genome of negative strand RNA viruses is not infectious and recovery of infectious virus from cDNA (“rescue”) requires intracellular co-expression of viral NP (or N), P and L proteins, along with a full length positive sense, or negative sense, genome RNA transcript, all under control of the bacteriophage T7 RNA polymerase promoter (e.g., Schnell et al., 1994 EMBO J., 13:4195-4203; Lawson et al. 1995 Proc. Natl. Acad. Sci. USA, 92:4471-4481; Whelan et al., 1995 Proc. Natl. Acad. Sci. USA, 92:8388-8392. The T7 RNA polymerase has been supplied, for example, either by a co-infecting recombinant vaccinia virus (Fuerst et al., 1986 Proc. Natl. Acad. Sci. USA, 83:8122-8126; Wyatt et al., 1995 Virology, 210:202-5), or by endogenous expression of T7 RNA polymerase in a transformed cell line (Radecke et al., 1995 EMBO J, 14:5773-5784).

The polymerase complex actuates and achieves transcription and replication by engaging the cis-acting signals at the 3′ end of the genome, in particular, the promoter region. Viral genes are then transcribed from the genome template unidirectionally from its 3′ to its 5′ end. There is generally less mRNA made from the downstream genes (e.g., the polymerase gene (L)) relative to their upstream neighbors (i.e., the nucleoprotein gene (NP)). Therefore, there is always a gradient of mRNA abundance according to the position of the genes relative to the 3′-end of the genome.

In rescue, after transfection of a genomic cDNA plasmid, an exact copy of genome RNA is produced by the combined action of phage T7 RNA polymerase and a vector-encoded ribozyme sequence that cleaves the RNA to form the 3′ termini. This RNA is packaged and replicated by viral proteins initially supplied by co-transfected expression plasmids. A specific method of rescue of mumps virus from cDNA is described in Clarke et al., J. Virol. 2000 J. Virol., 74:831-4838. This methodology has enabled targeted alteration of the MuV genome to develop rMuV as a vector for immunization can be used to insert nucleotide sequences encoding antigens so as to be able to express an antigen such as HIV-1 gag.

The boosting immunogenic composition used in this invention comprises a recombinant attenuated vesicular stomatitis virus (VSV). VSV is a member of the Rhabdoviridae family, has a non-segmented, negative-sense, single-stranded RNA genome. Its eleven kilobase (kb) genome has five genes which encode five structural proteins of the virus; the nucleocapsid protein (N), which is required in stoichiometric amounts for encapsidation of the replicated RNA; the phosphoprotein (P), which is a cofactor of the RNA-dependent RNA polymerase (L); the matrix protein (M) and the attachment glycoprotein (G) (Gallione et al. 1981, Rose and Gallione, 1981; Rose and Schubert, 1987 and Schubert et al., 1985.

VSV is an arthropod borne virus that can be transmitted to a variety of mammalian hosts, most commonly cattle, horses, swine and rodents. VSV infection of humans is uncommon, and in general is either asymptomatic or characterized by mild flu-like symptoms that resolve in three to eight days without complications. Because VSV is not considered a human pathogen, and pre-existing immunity to VSV is rare in the human population, the development of VSV derived vectors has been a focus in areas such as immunogenic compositions and gene therapy. For example, studies have established that VSV can serve as a highly effective vector for immunogenic compositions, expressing influenza virus haemagglutinin (Roberts et al., 1999 J. Virol., 73:3723-3732), measles virus H protein (Schlereth et al., 2000 J. Virol. 74:4652-4657) and HIV-1 env and gag proteins (Rose et al., 2001 Cell, 106:539-549). Other characteristics of VSV that render it an attractive vector include: (a) the ability to replicate robustly in cell culture; (b) the inability to either integrate into host cell DNA or undergo genetic recombination; (c) the existence of multiple serotypes, allowing for prime-boost immunization strategies; (d) foreign/heterologous genes of interest can be inserted into the VSV genome and expressed abundantly by the viral transcriptase; and (e) the development of a highly specialized system for the rescue of infectious virus from a cDNA copy of the virus genome (U.S. Pat. No. 6,033,886; U.S. Pat. No. 6,168,943).

Although there is little evidence of VSV neurological involvement during natural infection, animals (e.g., primates, rodents, herd animals) that are inoculated intracerebrally (and in the case of rodents intranasally) with wild-type virus, mouse brain passaged wild-type virus or cell culture adapted wild-type virus, can develop clinical signs of disease, and usually die two to eight days post inoculation. Because of these observations, and the need to produce a vector for immunogenic compositions for use in humans that has an exceptional safety profile, VSV vectors under development are tested in stringent, primate and small animal neurovirulence models. These tests are designed to detect any residual virulence in attenuated VSV vectors before consideration for advancement to human clinical trials.

The attenuation of prototypic-VSV vectors resulted from the accumulation of multiple nucleotide substitutions throughout the virus genome during serial passage in vitro and the synthesis and assembly of the genome cDNA. These mutations had pleiotropic effects that rendered the virus less pathogenic in mice than the lab-adapted virus from which it was derived (Roberts et al., 1998). Prototypic further attenuated VSV vectors were also developed by truncation of the cytoplasmic tail region of the virus G protein, leading to VSV mutants that were defective in budding from the plasma membrane of infected cells (Schnell et al., 1998).

As is the case in producing recombinant RNA viruses, similar in nature to the mumps virus mentioned above, a rescue method is undertaken. A live VSV may be isolated and rescued using techniques known in the art. Exemplary rescue methods for VSV are described in U.S. Pat. No. 6,033,886, U.S. Pat. No. 6,596,529 and WO 2004/113517, U.S. Pat. Nos. 6,044,886; 6,168,943; and 5,789,299; International Patent Publication No. WO99/02657, Conzelmann, 1998, Ann. Rev. Genet., 32:123-162; Roberts and Rose, 1998, Virol., 247:1-6; Lawson et al, 1995 Proc. Natl. Acad. Sci., USA 92:4477-4481, each incorporated herein by reference. A cloned DNA equivalent of the VSV genome is placed between a suitable DNA-dependent RNA polymerase promoter (e.g., the T7 RNA polymerase promoter) and a self-cleaving ribozyme sequence (e.g., the hepatitis delta ribozyme), which is inserted into a suitable transcription vector (e.g., a propagatable bacterial plasmid). This transcription vector provides the readily manipulatable DNA template from which the RNA polymerase (e.g., T7 RNA polymerase) can faithfully transcribe a single-stranded RNA copy of the VSV antigenome (or genome) with the precise, or nearly precise, 5′ and 3′ termini. The orientation of the VSV genomic DNA copy and the flanking promoter and ribozyme sequences determine whether antigenome or genome RNA equivalents are transcribed. Also required for rescue of new VSV progeny are the VSV-specific trans-acting support proteins needed to encapsidate the naked, single-stranded VSV antigenome or genome RNA transcripts into functional nucleocapsid templates: the viral nucleocapsid (N) protein, the polymerase-associated phosphoprotein (P) and the polymerase (L) protein. These proteins comprise the active viral RNA-dependent RNA polymerase which must engage this nucleocapsid template to achieve transcription and replication. Any suitable VSV strain or serotype may be used according to the present invention, including, but not limited to, VSV Indiana, VSV New Jersey, VSV Chandipura, VSV San Juan, VSV Glasgow, and the like.

VSV genomes have been shown to accommodate more than one foreign gene, with expansion to at least three kilobases. The genomes of these viruses are very stable, do not undergo recombination, and rarely incur significant mutations. In addition, since their replication is cytoplasmic, and their genomes are comprised of RNA, they are incapable of integrating within the genomes of infected host cells. Also, these negative-strand RNA viruses possess relatively simple transcriptional control sequences, which are readily manipulatable for efficient foreign gene insertion. Finally, the level of foreign gene expression can be modulated by changing the position of the foreign gene relative to the viral transcription promoter. The 3′ to 5′ gradient of gene expression reflects the decreasing likelihood that the transcribing viral polymerase will traverse successfully each intergenic gene stop/gene start signal encountered as it progresses along the genome template. Thus, foreign genes placed in proximity to a 3′ terminal transcription initiation promoter are expressed abundantly, while those inserted in more distal genomic positions, are less so.

VSV replicates to high titers in a large array of different cell types, and viral proteins are expressed in great abundance. This not only means that VSV will act as a potent functional foreign gene delivery vehicle, but also, that relevant rVSV vectors can be scaled to manufacturing levels in cell lines approved for the production of human biologicals.

The rVSV has the capacity to deliver foreign/heterologous genes encoding critical protective immunogens from viral pathogens to a broad array of different cell types, and to subsequently cause the abundant expression of authentically-configured immunogenic proteins (Haglund, K., et al, 2000 Virol., 268:112-21; Kahn, J. S. et al, 1999 Virol., 254:81-91; Roberts, A. et al, 1999 J. Virol., 73:3723-32; Rose, N. F. et al, 2000 J. Virol., 74:10903-10; and Schlereth, B. et al. 2000 J. Virol., 74:4652-7). The immunogens, so expressed, simultaneously elicit both highly durable virus neutralizing antibody responses, as well as protective cytotoxic T lymphocytes (CTL) (Roberts, A. et al, 1998 J. Virol., 72:4704-11).

Live VSV vectors are safe because wild-type VSV produces little to no disease symptoms or pathology in healthy humans, even in the face of substantial virus replication (Tesh, R. B. et al, 1969 Am. J. Epidemiol., 90:255-61). Additionally human infection with, and thus pre-existing immunity to VSV is rare. Given further attenuation, these rVSV compositions are suitable for use in immunocompromised or otherwise less robust human subjects. A significant advantage of use of the VSV vector in this method is that a number of serotypes of VSV exist due to the exchange or modification of the viral attachment protein G of the VSV. Thus, different serotypes of VSV vector carrying the same heterologous antigen can be used for repeated administration to avoid any interfering neutralizing antibody response generated to the VSV G protein by the host's immune system.

A recombinant VSV can be designed using techniques previously described in the art, which carries the selected antigen and its regulatory sequences inserted into any position of the VSV under the control of the viral transcription promoter. In one embodiment, the heterologous gene encoding the selected antigen is inserted between the G and L coding regions of VSV. In another embodiment, the heterologous gene may be fused in the site of the G protein. In still other embodiments, the heterologous gene is fused to the site, or adjacent to, any of the other VSV genes.

In still other embodiments, the genes are translocated or ‘shuffled’ to different positions in the genome. In particular, the N gene is ‘shuffled’ to different distal positions in the genome. The cloning strategy used to create these plasmids employs a method described by Ball, L. A. et al. 1999 J. Virol., 73:4705-12. This technique takes advantage of the fact that the gene-end/gene-start signals found between each coding sequence are nearly identical, and allows gene rearrangements to be constructed without introducing any nucleotide substitutions. Alternatively, a few strategic point mutations may be introduced into noncoding sequences to create convenient restriction sites that facilitate genome rearrangements.

In still further embodiments of these vectors, the carboxy-terminal coding sequence for the 29 amino acid cytoplasmic domain of the G gene is truncated by deleting amino acids from the 5° C terminus of the G gene. Alternatively, the G gene is deleted entirely. In one embodiment, the entire cytoplasmic domain of the G gene is removed. In another embodiment at least 28 amino acids of the cytoplasmic domain are removed. In still a further embodiment, about 20 amino acids of the cytoplasmic domain are deleted. In still a further embodiment, about 10 or fewer amino acids of the cytoplasmic domain are deleted.

Both the shuffled genome approach and the G protein modification approach reportedly generate partial growth defects (Flanagan, E. B., et al 2001 J. Virol. 75:6107-14; Schnell, M. J. et al. 1998 EMBO J., 17:1289-96). It is anticipated that such modifications of the VSV may lead to a more attenuated phenotype or even to a non-replicating VSV for use in various embodiments of the present invention. See, e.g., Johnson, J. E. et al, 1998 Virol., 251:244-52.37; Johnson, J. E., et al., 1997 J. Virol., 71:5060-8.

In some embodiments of the first or second immunogenic compositions herein, the selected antigen is an HIV-1 gag and/or env (gp160). In still other embodiments, the antigen is an HIV-1 pol, net, vpr, vpu, vif or tat gene. Preferably the gene sequence encoding the antigen is optimized, such as by codon selection appropriate to the intended host and/or by removal of any inhibitory sequences, also discussed below with regard to antigen preparation.

To overcome any potential problem of diminished vector replication efficiencies with sequential administration, a vector set of similar design, each carrying a G gene from a different VSV serotype, permits successful booster immunizations. The primary amino acid sequences of the G proteins from VSV Indiana, New Jersey, and Chandipura, are sufficiently divergent such that preexisting immunity to one does not preclude infection and replication of the others. Thus, the neutralizing antibody response generated by rVSV (Indiana) should not interfere with replication of either rVSV (New Jersey) or rVSV (Chandipura). A vector set that can permit successful sequential immunizations can be prepared by replacing the G gene from VSV Indiana with either the divergent homolog from VSV Chandipura or from VSV New Jersey, forming three immunologically distinct vectors.

This rVSV immunogenic composition may include therefore one rVSV encoding a single selected antigen for expression in the host. According to the present method, the rVSV immunogenic composition comprises one rVSV comprising a nucleic acid sequence encoding one or more copies of the same selected antigen. Alternatively, the composition may contain one rVSV expressing multiple selected antigens. Each antigen may be under the control of separate regulatory elements or components. Alternatively, each antigen may be under the control of the same regulatory elements. In still another embodiment, the rVSV composition may contain multiple rVSVs, wherein each rVSV encodes the same or a different antigen.

Previously utilized VSV compositions, putatively attenuated or not, have had unacceptable levels of residual virulence when tested in small animal and non-human primate neurovirulence models. The development of a VSV vector for uses such as a vector for immunogenic compositions or a gene therapy vector has required VSV vectors to have minimal to non-detectable levels of pathogenicity in animal neurovirulence models. The viral vector developed and subsequently utilized in this invention, rVSVN4CT1, was highly attenuated by a combination of specific N gene translocations and G gene truncations (see Clarke et al., 2007 J. Virol., 81: 2056-64). For this invention, the HIV-1 gag gene was inserted into the first position of the genome, the N gene is in position 4, and the G protein is expressed with a single amino acid in the cytoplasmic tail. This recombinant virus containing this vector proved to be avirulent in mouse models following intracranial inoculation.

The antigenic or immunogenic compositions useful in the methods and compositions of this invention enhance the immune response in a vertebrate host to a selected antigen. The selected antigen may be a protein, polypeptide, peptide, fragment or a fusion thereof derived from a pathogenic virus, bacterium, fungus or parasite. Alternatively, the selected antigen may be a protein, polypeptide, peptide, fragment or fusion thereof derived from a cancer cell or tumor cell. In some embodiments, the selected antigen may be a protein, polypeptide, peptide, fragment or fusion thereof derived from an allergen so as to interfere with the production of IgE so as to moderate allergic responses to the allergen. In some embodiments, the selected antigen may be a protein, polypeptide, peptide, fragment or fusion thereof derived from a molecule or portion thereof which represents those produced by a host (a self molecule) in an undesired manner, amount or location, such as those from amyloid precursor protein, so as to prevent or treat disease characterized by amyloid deposition in a vertebrate host. In one embodiment of this invention, the selected antigen is a protein, polypeptide, peptide or fragment derived from HIV-1.

The invention is also directed to methods for increasing the ability of an immunogenic composition containing a selected antigen (1) from a pathogenic virus, bacterium, fungus or parasite to elicit the immune response of a vertebrate host, or (2) from a cancer antigen or tumor-associated antigen from a cancer cell or tumor cell to elicit a therapeutic or prophylactic anti-cancer effect in a vertebrate host, or (3) from an allergen so as to interfere with the production of IgE so as to moderate allergic responses to the allergen, or (4) from a molecule or portion thereof which represents those produced by a host (a self molecule) in an undesired manner, amount or location, so as to reduce such an undesired effect.

In another embodiment, immunogenic compositions utilizing the prime/boost regimen of this invention include those directed to the prevention and/or treatment of disease determined by the presence of, without limitation an HIV antigen, an HTLV antigen, an SIV antigen, an RSV antigen, a PIV antigen, an HSV antigen, a CMV antigen, an Epstein-Barr virus antigen, a Varicella-Zoster virus antigen, a mumps virus antigen, a measles virus antigen, an influenza virus antigen, a poliovirus antigen, a rhinovirus antigen, a hepatitis A virus antigen, a hepatitis B virus antigen, a hepatitis C virus antigen, a Norwalk virus antigen, a togavirus antigen, an alphavirus antigen, a rubella virus antigen, a rabies virus antigen, a Marburg virus antigen, an Ebola virus antigen, a papilloma virus antigen, a polyoma virus antigen, a metapneumovirus antigen, a coronavirus antigen, a Vibrio cholerae antigen, a Plasmodium falciparum antigen, a Plasmodium vivax antigen, a Plasmodium ovale antigen, a Plasmodium malariae antigen, a Plasmodium knowlesi antigen, a Streptococcus pneumoniae antigen, Streptococcus pyogenes antigen, a Helicobacter pylori antigen, a Streptococcus agalactiae antigen, a Neisseria meningitidis antigen, a Neisseria gonorrheae antigen, a Corynebacteria diphtheriae antigen, a Clostridium tetani antigen, a Bordetella pertussis antigen, a Haemophilus antigen, a Chlamydia antigen, a Escherichia coli antigen, a cytokine, and a T-helper epitope and a CTL epitope.

In another embodiment, immunogenic compositions against fungal pathogens utilizing the prime/boost regimen of this invention include those directed to the prevention and/or treatment of disease caused by, without limitation, Aspergillis, Blastomyces, Candida, Coccidiodes, Cryptococcus and Histoplasma.

In another embodiment, immunogenic compositions against parasites utilizing the prime/boost regimen of this invention include those directed to the prevention and/or treatment of disease caused by, without limitation, Leishmania major, Ascaris, Trichuris, Giardia, Schistosoma, Plasmodium, Cryptosporidium, Trichomonas, Toxoplasma gondii and Pneumocystis carinii.

In another embodiment, immunogenic compositions for eliciting a therapeutic or prophylactic anti-cancer effect in a vertebrate host, which utilize the prime/boost regimen of this invention include those utilizing a cancer antigen or tumor-associated antigen, including, without limitation, prostate specific antigen, carcino-embryonic antigen, MUC-1, Her2, CA-125 and MAGE-3.

Desirable immunogenic compositions for moderating responses to allergens in a vertebrate host, which utilize the prime/boost regimen of this invention include those containing an allergen or fragment thereof. Examples of such allergens are described in U.S. Pat. No. 5,830,877 and International Patent Publication No. WO99/51259, which are hereby incorporated by reference. Such allergens include, without limitation, pollen, insect venoms, animal dander, fungal spores and drugs (such as penicillin). These immunogenic compositions interfere with the production of IGE antibodies, a known cause of allergic reactions.

It is also desirable in selection and use of the antigenic sequences for design of the rVSV and rMuV compositions of this invention to alter codon usage of the selected antigen-encoding gene sequence, as well as the DNA plasmids into which they are inserted, and/or to remove inhibitory sequences therein. The removal of inhibitory sequences can be accomplished by using the technology discussed in detail in U.S. Pat. Nos. 5,965,726; 5,972,596; 6,174,666; 6,291,664; and 6,414,132; and in International Patent Publication No. WO01/46408, incorporated by reference herein. Briefly described, this technology involves mutating identified inhibitor/instability sequences in the selected gene, preferably with multiple point mutations.

In one embodiment, the immunogenic compositions of this invention desirably employ one or more sequences optimized to encode HIV-1 antigens, such as the gag, pol and nef antigens, or immunogenic fragments or fusions thereof.

Suitable promoters for use in any of the components of this invention may be readily selected from among constitutive promoters, inducible promoters, tissue-specific promoters and others. Examples of constitutive promoters that are non-specific in activity and employed in the nucleic acid molecules encoding an antigen of this invention include, without limitation, the retroviral Rous sarcoma virus (RSV) promoter, the retroviral LTR promoter (optionally with the RSV enhancer), the cytomegalovirus (CMV) promoter (optionally with the CMV enhancer) (see, e.g., Boshart et al, 1985 Cell, 41:521-530), the SV40 promoter, the dihydrofolate reductase promoter, the β-actin promoter, the phosphoglycerol kinase (PGK) promoter, and the EF1α promoter (Invitrogen). Inducible promoters that are regulated by exogenously supplied compounds, include, without limitation, the arabinose promoter, the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al, 1996 Proc. Natl. Acad. Sci. USA, 93:3346-3351), the tetracycline-repressible system (Gossen et al, 1992 Proc. Natl. Acad. Sci. USA, 89:5547-5551), the tetracycline-inducible system (Gossen et al, 1995 Science, 268:1766-1769, see also Harvey et al, 1998 Curr. Opin. Chem. Biol., 2:512-518), the RU486-inducible system (Wang et al, 1997 Nat. Biotech., 15:239-243 and Wang et al, 1997 Gene Ther., 4:432-441) and the rapamycin-inducible system (Magari et al, 1997 J. Clin. Invest., 100: 2865-2872).

Other types of inducible promoters that may be useful in this context are those regulated by a specific physiological state, e.g., temperature or acute phase or in replicating cells only. Useful tissue-specific promoters include the promoters from genes encoding skeletal β-actin, myosin light chain 2A, dystrophin, muscle creatine kinase, as well as synthetic muscle promoters with activities higher than naturally-occurring promoters (see Li et al., 1999 Nat. Biotech., 17:241-245). Examples of promoters that are tissue-specific are known for the liver (albumin, Miyatake et al. 1997 J. Virol., 71:5124-32; hepatitis B virus core promoter, Sandig et al., 1996 Gene Ther., 3: 1002-9; alpha-fetoprotein (AFP), Arbuthnot et al., 1996 Hum. Gene Ther., 7:1503-14), bone (osteocalcin, Stein et al., 1997 Mol. Biol. Rep., 24:185-96; bone sialoprotein, Chen et al., 1996 J. Bone Miner. Res., 11:654-64), lymphocytes (CD2, Hansal et al., 1988 J. Immunol., 161:1063-8; immunoglobulin heavy chain; T cell receptor α chain), neuronal (neuron-specific enolase (NSE) promoter, Andersen et al. 1993 Cell. Mol. Neurobiol., 13:503-15; neurofilament light-chain gene, Piccioli et al., 1991 Proc. Natl. Acad. Sci. USA, 88:5611-5; the neuron-specific ngf gene, Piccioli et al., 1995 Neuron, 15:373-84); among others. See, e.g., International Patent Publication No. WO00/55335 for additional lists of known promoters useful in this context.

As discussed below in some detail, in some embodiments, the first or second immunogenic compositions may further contain or be administered with an adjuvant such as a cytokine, a lymphokine or a genetic adjuvant. The cytokine may be administered as a protein or be encoded by insertion of the cytokine encoding sequence in a recombinant viral vector. For example, the cytokine encoding sequence may be inserted into any position in the VSV genome and expressed from the viral transcription promoter. In the embodiments exemplified in this invention, a desirable cytokine for administration with the first or second immunogenic composition of this invention is Interleukin-12. In some embodiments, the rVSV composition includes an additional recombinant virus encoding a selected cytokine. In still another embodiment, the rVSV includes a sequence expressing a cytokine, e.g., IL-12 present in the same rVSV as is expressing the antigen.

Desirably each subsequent rVSV composition has a different serotype, but the same antigen encoding sequence. The different serotypes are selected from among known naturally occurring serotypes and from among any synthetic serotypes provided by manipulation of the VSV G protein. Among known methods for altering the G protein of rVSV are the technology described in International Publication No. WO99/32648 and Rose, N. F. et al. 2000 J. Virol., 74:10903-10.

The immunogenic compositions used in this invention can further comprise an immunologically acceptable diluent. The immunogenic compositions may also be mixed with such diluents in a conventional manner. As used herein the language “pharmaceutically acceptable carrier” is intended to include any and all solvents, dispersion media, coatings, antibacterial and antifungal agents, isotonic and absorption delaying agents, and the like, compatible with administration to humans or other vertebrate hosts. The appropriate carrier is evident to those skilled in the art and will depend in large part upon the route of administration.

Still additional components that may be present in the immunogenic compositions of this invention are adjuvants, preservatives, surface active agents, and chemical stabilizers, suspending or dispersing agents. Typically, stabilizers, adjuvants, and preservatives are optimized to determine the best formulation for efficacy in the target human or animal.

An adjuvant is a substance that enhances the immune response when administered together with an immunogenic composition. A number of cytokines or lymphokines have been shown to have immune modulating activity, and thus may be used as adjuvants, including, but not limited to, the interleukins 1-α, 1-β, 2, 4, 5, 6, 7, 8, 10, 12 (see, e.g., U.S. Pat. No. 5,723,127), 13, 14, 15, 16, 17 and 18 (and its mutant forms), the interferons-α,β and γ, granulocyte-macrophage colony stimulating factor (see, e.g., U.S. Pat. No. 5,078,996 and ATCC Accession Number 39900), macrophage colony stimulating factor, granulocyte colony stimulating factor, GSF, and the tumor necrosis factors α and β. Still other adjuvants useful in this invention include a chemokine, including without limitation, MCP-1, MIP-1α, MIP-1β, and RANTES. Adhesion molecules, such as a selectin, e.g., L-selectin, P-selectin and E-selectin may also be useful as adjuvants. Still other useful adjuvants include, without limitation, a mucin-like molecule, e.g., CD34, GlyCAM-1 and MadCAM-1, a member of the integrin family such as LFA-1, VLA-1, Mac-1 and p150.95, a member of the immunoglobulin superfamily such as PECAM, ICAMs, e.g., ICAM-1, ICAM-2 and ICAM-3, CD2 and LFA-3, co-stimulatory molecules such as CD40 and CD40L, growth factors including vascular growth factor, nerve growth factor, fibroblast growth factor, epidermal growth factor, B7.2, PDGF, BL-1, and vascular endothelial growth factor, receptor molecules including Fas, TNF receptor, FIt, Apo-1, p55, WSL-1, DR3, TRAMP, Apo-3, AIR, LARD, NGRF, DR4, DR5, KILLER, TRAIL-R2, TRICK2, and DR6. Still another adjuvant molecule includes Caspase (ICE). See, also International Patent Publication Nos. WO98/17799 and WO99/43839, incorporated herein by reference.

Suitable adjuvants used to enhance an immune response include, without limitation, MPL™ (3-O-deacylated monophosphoryl lipid A; Corixa, Hamilton, Mont.), which is described in U.S. Pat. No. 4,912,094, which is hereby incorporated by reference. Also suitable for use as adjuvants are synthetic lipid A analogs or aminoalkyl glucosamine phosphate compounds (AGP), or derivatives or analogs thereof, which are available from Corixa (Hamilton, MT), and which are described in U.S. Pat. No. 6,113,918, which is hereby incorporated by reference. One such AGP is 2-[(R)-3-Tetradecanoyloxytetradecanoylamino] ethyl 2-Deoxy-4-O-phosphono-3-O-[(R)-3-tetradecanoyoxytetradecanoyl]-2-[(R)-3-tetradecanoyloxytetradecanoyl-amino]-b-D-glucopyranoside, which is also known as 529 (formerly known as RC529). This 529 adjuvant is formulated as an aqueous form or as a stable emulsion.

Still other adjuvants include mineral oil and water emulsions, aluminum salts (alum), such as aluminum hydroxide, aluminum phosphate, etc., Amphigen, Avridine, L121/squalene, D-lactide-polylactide/glycoside, pluronic polyols, muramyl dipeptide, killed Bordetella, saponins, such as Stimulon™ QS-21 (Antigenics, Framingham, Mass.), described in U.S. Pat. No. 5,057,540, which is hereby incorporated by reference, and particles generated therefrom such as ISCOMS (immunostimulating complexes), Mycobacterium tuberculosis, bacterial lipopolysaccharides, synthetic polynucleotides such as oligonucleotides containing a CpG motif (U.S. Pat. No. 6,207,646, which is hereby incorporated by reference), a pertussis toxin (PT), or an E. coli heat-labile toxin (LT), particularly LT-K63, LT-R72, PT-K9/G129; see, e.g., International Patent Publication Nos. WO 93/13302 and WO 92/19265, incorporated herein by reference.

Also useful as adjuvants are cholera toxins and mutants thereof, including those described in published International Patent Application number WO 00/18434 (wherein the glutamic acid at amino acid position 29 is replaced by another amino acid, other than aspartic acid, preferably a histidine). Similar CT toxins or mutants are described in published International Patent Application number WO 02/098368 (wherein the isoleucine at amino acid position 16 is replaced by another amino acid, either alone or in combination with the replacement of the serine at amino acid position 68 by another amino acid; and/or wherein the valine at amino acid position 72 is replaced by another amino acid). Other CT toxins are described in published International Patent Application number WO 02/098369 (wherein the arginine at amino acid position 25 is replaced by another amino acid; and/or an amino acid is inserted at amino acid position 49; and/or two amino acids are inserted at amino acid positions 35 and 36).

Other additives can be included in the immunogenic compositions of this invention, including preservatives, stabilizing ingredients, surface active agents, and the like. Suitable exemplary preservatives include chlorobutanol, potassium sorbate, sorbic acid, sulfur dioxide, propyl gallate, the parabens, ethyl vanillin, glycerin, phenol, and parachlorophenol. Suitable stabilizing ingredients that may be used include, for example, casamino acids, sucrose, gelatin, phenol red, N-Z amine, monopotassium diphosphate, lactose, lactalbumin hydrolysate, and dried milk. Suitable surface active substances include, without limitation, quinone analogs, hexadecylamine, octadecylamine, octadecyl amino acid esters, lysolecithin, dimethyl-dioctadecylammonium bromide), methoxyhexadecylgylcerol, and pluronic polyols; polyamines, e.g., pyran, dextransulfate, poly IC, carbopol; peptides, e.g., muramyl peptide and dipeptide, dimethylglycine, tuftsin; oil emulsions; and mineral gels, e.g., aluminum phosphate, etc. and immune stimulating complexes (ISCOMS). The immunogenic compositions may also be incorporated into liposomes for use as an immunogenic composition and may also contain other additives suitable for the selected mode of administration of the composition. The composition of the invention may also involve lyophilized polynucleotides, which can be used with other pharmaceutically acceptable excipients for developing powder, liquid or suspension dosage forms. See, e.g., Remington: The Science and Practice of Pharmacy, Vol. 2, 19^{th} edition (1995), e.g., Chapter 95 Aerosols; and International Patent Publication No. WO99/45966, the teachings of which are hereby incorporated by reference.

The immunogenic compositions of the present invention can contain additives suitable for administration via any conventional route of administration. The immunogenic compositions of the invention can be prepared for administration to subjects in the form of, for example, liquids, powders, aerosols, tablets, capsules, enteric-coated tablets or capsules, or suppositories. Thus, the immunogenic compositions may also include, but are not limited to, suspensions, solutions, emulsions in oily or aqueous vehicles, pastes, and implantable sustained-release or biodegradable formulations. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition. Other useful parenterally-administrable formulations include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Other additives can be included in the immunogenic compositions of this invention, including preservatives, stabilizing ingredients, surface active agents, and the like.

Suitable exemplary preservatives include chlorobutanol, potassium sorbate, sorbic acid, sulfur dioxide, propyl gallate, the parabens, ethyl vanillin, glycerin, phenol, and parachlorophenol.

Suitable stabilizing ingredients that may be used include, for example, casamino acids, sucrose, gelatin, phenol red, N-Z amine, monopotassium diphosphate, lactose, lactalbumin hydrolysate, and dried milk.

Suitable surface active substances include, without limitation, quinone analogs, hexadecylamine, octadecylamine, octadecyl amino acid esters, lysolecithin, dimethyl-dioctadecylammonium bromide), methoxyhexadecylgylcerol, and pluronic polyols; polyamines, e.g., pyran, dextransulfate, poly IC, carbopol; peptides, e.g., muramyl peptide and dipeptide, dimethylglycine, tuftsin; oil emulsions; and mineral gels, e.g., aluminum phosphate, etc. and immune stimulating complexes (ISCOMS). The plasmids, rMuVs and rVSVs may also be incorporated into liposomes for use as an immunogenic composition. The immunogenic compositions may also contain other additives suitable for the selected mode of administration of the composition. The composition of the invention may also involve lyophilized polynucleotides, which can be used with other pharmaceutically acceptable excipients for developing powder, liquid or suspension dosage forms. See, e.g., Remington: The Science and Practice of Pharmacy, Vol. 2, 19^{th} edition (1995), e.g., Chapter 95 Aerosols; and International Patent Publication No. WO99/45966, the teachings of which are hereby incorporated by reference.

The immunogenic compositions of the present invention can contain additives suitable for administration via any conventional route of administration. In some preferred embodiments, the immunogenic composition of the invention is prepared for administration to human subjects in the form of, for example, liquids, powders, aerosols, tablets, capsules, enteric-coated tablets or capsules, or suppositories. Thus, the immunogenic compositions may also include, but are not limited to, suspensions, solutions, emulsions in oily or aqueous vehicles, pastes, and implantable sustained-release or biodegradable formulations. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition. Other useful parenterally administrable formulations include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

The immunogenic compositions of the present invention are not limited by the selection of the conventional, physiologically acceptable, carriers, adjuvants, or other ingredients useful in pharmaceutical preparations of the types described above. The preparation of these pharmaceutically acceptable compositions, from the above-described components, having appropriate pH isotonicity, stability and other conventional characteristics is within the skill of the art.

In general, selection of the appropriate “effective amount” or dosage for the components of the immunogenic compositions of the present invention will also be based upon the identity of the antigen in the immunogenic composition(s) employed, as well as the physical condition of the subject, most especially including the general health, age and weight of the immunized subject. The method and routes of administration and the presence of additional components in the immunogenic compositions may also affect the dosages and amounts of the compositions. Such selection and upward or downward adjustment of the effective dose is within the skill of the art. The amount of composition required to induce an immune response, preferably a protective response, or produce an exogenous effect in the subject without significant adverse side effects varies depending upon these factors. Suitable doses of the immunogenic compositions of the present invention are readily determined by persons skilled in the art.

The immunogenic compositions of this invention are administered to a human or to a non-human vertebrate by a variety of routes including, but not limited to, intravenous, intradermal, subcutaneous, intramuscular, intraperitoneal, oral, rectal, intranasal, buccal, vaginal and ex vivo. The appropriate route is selected depending on the nature of the immunogenic composition used, and an evaluation of the age, weight, sex and general health of the patient and the antigens present in the immunogenic composition, and similar factors by an attending physician. The selection of dosages and routes of administration are not limitations upon this invention.

Similarly, the order of immunogenic composition administration and the time periods between individual administrations may be selected by one of skill in the art based upon the physical characteristics and precise responses of the host to the application of the method. Such optimization is expected to be well within the skill of the art.

In still another embodiment, the present invention provides a pharmaceutical kit for ready administration of an immunogenic, prophylactic, or therapeutic regimen for treatment of any of the above-noted diseases or conditions for which an immune response to an antigen is desired. This kit is designed for use in a method of inducing a high level of antigen-specific immune response in a mammalian or vertebrate subject. The kit contains at least one immunogenic composition comprising an rMuV comprising a nucleic acid sequence encoding an antigen under the control of regulatory sequences directing expression thereof in a mammalian or vertebrate cell. Preferably multiple prepackaged dosages of the immunogenic composition are provided in the kit for multiple administrations. The kit also contains at least one immunogenic composition comprising a vesicular stomatitis virus (rVSV) comprising a nucleic acid sequence encoding the same antigen under the control of regulatory sequences directing expression thereof in a mammalian or vertebrate cell. Preferably multiple prepackaged dosages of the rVSV immunogenic composition are provided in the kit for multiple administrations.

Where the above-described immunogenic compositions do not also contain DNA plasmids and/or rVSV or rMuV that express a cytokine, such as IL-12, the kit also optionally contains a separate cytokine composition or multiple prepackaged dosages of the cytokine composition for multiple administrations. These cytokine compositions are generally nucleic acid compositions comprising a DNA sequence encoding the selected cytokine under the control of regulatory sequences directing expression thereof in a mammalian or vertebrate cell.

The kit also contains instructions for using the immunogenic compositions in a prime/boost method as described herein. The kits may also include instructions for performing certain assays, various carriers, excipients, diluents, adjuvants and the like above-described, as well as apparatus for administration of the compositions, such as syringes, spray devices, etc. Other components may include disposable gloves, decontamination instructions, applicator sticks or containers, among other compositions.

As demonstrated in the Examples below, a prime/boost protocol of this invention induces in the immunized subject a synergistic effect on antigen-specific cellular immune responses. In fact, when these responses induced by a prime/boost protocol of this invention are compared to the results of administering the priming and boosting composition in a reverse order, the nature of the response to the compositions of this invention is dramatically evident.

### Definitions

As used in the specification and the appended claims, the singular forms “a,” “an” and “the” include reference to the plural unless the context clearly dictates otherwise.

The term “about” means within 20%, more preferably within 10% and more preferably within 5%.

The term “antigen” refers to a compound, composition, or immunogenic substance that can stimulate the production of antibodies or a T-cell response, or both, in an animal, including compositions that are injected or absorbed into an animal. The immune response may be generated to the whole molecule, or to a portion of the molecule (e.g., an epitope or hapten). The term may be used to refer to an individual macromolecule or to a homogeneous or heterogeneous population of antigenic macromolecules. An antigen reacts with the products of specific humoral and/or cellular immunity. The term “antigen” broadly encompasses moieties including proteins, polypeptides, antigenic protein fragments, nucleic acids, oligosaccharides, polysaccharides, organic or inorganic chemicals or compositions, and the like. The term “antigen” includes all related antigenic epitopes. Epitopes of a given antigen can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N. J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Furthermore, for purposes of the present invention, an “antigen” can also include includes modifications, such as deletions, additions and substitutions (generally conservative in nature, but they may be non-conservative), to the native sequence, so long as the protein maintains the ability to elicit an immunological response. These modifications may be deliberate, as through site-directed mutagenesis, or through particular synthetic procedures, or through a genetic engineering approach, or may be accidental, such as through mutations of hosts, which produce the antigens. Furthermore, the antigen can be derived or obtained from any virus, bacterium, parasite, protozoan, or fungus, and can be a whole organism. Similarly, an oligonucleotide or polynucleotide, which expresses an antigen, such as in nucleic acid immunization applications, is also included in the definition. Synthetic antigens are also included, for example, polyepitopes, flanking epitopes, and other recombinant or synthetically derived antigens (Bergmann et al. (1993) Eur. J. Immunol. 23:2777 2781; Bergmann et al. (1996) J. Immunol. 157:3242 3249; Suhrbier, A. (1997) Immunol. and Cell Biol. 75:402 408; Gardner et al. (1998) 12th World AIDS Conference, Geneva, Switzerland, Jun. 28 Jul. 3,1998).

The term “attenuated” refers to a strain of pathogen whose pathogenicity has been reduced so that it will initiate an immune response without producing the specific disease. An attenuated strain of a virus is less virulent than the parental strain from which it is derived. Conventional means are used to introduce attenuating mutations to generate a modified virus, such as chemical mutagenesis during virus growth in cell cultures to which a chemical mutagen has been added. An alternative means of introducing attenuating mutations comprises making pre-determined mutations using site-directed mutagenesis. One or more mutations may be introduced. These viruses are then screened for attenuation of their biological activity in cell culture and/or in an animal model. If the attenuated phenotype of the rescued virus is present, challenge experiments can be conducted with an appropriate animal model. Non-human primates can serve as an appropriate animal model for the pathogenesis of human disease. These primates are first immunized with the attenuated, recombinantly-produced virus, then challenged with the wild-type form of the virus.

The term “boosting an immune response to an antigen” refers to the administration to a subject with a second, boosting immunogenic composition after the administration of the priming immunogenic composition. In one embodiment, the boosting administration of the immunogenic composition is given about 2 to 27 weeks after administration of the priming dose

The term “cell mediated immunity” refers to the primary immune response involving T-lymphocytes (T cells) that respond to introduction of foreign antigens. CD8+ T cells killer T cells induce cell death in cells that are infected by viruses or parasites. CD4+ T helper cells have no cytotoxic or phagocytic activity but direct other cells to clear pathogens and release cytokines. Cell mediated immunity does not require the production of antibodies.

The term “heterologous” refers to a combination of elements not naturally occurring. For example, heterologous antigen refers to an antigen not naturally in an organism, cell or virus. The heterologous antigen is encoded by DNA and may include a gene foreign to the organism, cell or virus. In the present invention, a heterologous rMuV or rVSV antigen refers to an antigen not naturally encoded by MuV or by VSV, but has been altered using techniques well known to one skilled in the art to include a nucleic acid that encodes a protein or antigen from a different source.

The term “immunogenic composition” refers to any pharmaceutical composition containing an antigen which composition can be used to elicit an immune response in a mammal. The immune response can include a T cell response, a B cell response, or both a T cell and B cell response. The composition may serve to sensitize the mammal by the presentation of antigen in association with MHC molecules at the cell surface. In addition, antigen-specific T-lymphocytes or antibodies can be generated to allow for the future protection of an immunized host. An immunogenic composition may contain a live, attenuatedvirus composition that induces either a cell-mediated (T cell) immune response or an antibody-mediated (B cell) immune response, or both.

The term “immune response” is meant to refer to any response to an antigen or antigenic determinant by the immune system of a vertebrate subject, including humoral immune responses (e.g. production of antigen-specific antibodies) and cell-mediated immune responses (e.g. lymphocyte proliferation).

The term “pharmaceutically acceptable carrier” is intended to include any and all solvents, dispersion media, coatings, antibacterial and antifungal agents, isotonic and absorption delaying agents, compatible with administration to humans or other vertebrate hosts. The appropriate carrier is evident to those skilled in the art and will depend in large part upon the route of administration. Additional components that may be present in this invention are adjuvants, preservatives, surface active agents, chemical stabilizers, suspending or dispersing agents. Typically, stabilizers, adjuvants and preservatives are optimized to determine the best formulation for efficacy in the target subject.

The term “priming an immune response to an antigen” refers to the administration to a subject with an immunogenic composition which induces a higher level of an immune response to the antigen upon subsequent administration with the same or a second composition, than the immune response obtained by administration with a single immunogenic composition.

The term “propagation incompetent”, as used herein, refers to an attenuated virus that is restricted to a single round of replication in vivo and is unable to spread beyond primary infected cells. As used herein, a propagation incompetent rVSV has a mutation in the G gene which normally encodes the transmembrane glycoprotein and facilitates virus attachment to a host cell. In some embodiments the entire G gene is deleted (VSVΔG); in other embodiments, the G protein ectodomain (G stem) is deleted (VSV-Gstem). Both the VSVΔG and VSV-Gstem deletions require the G protein to be provided in trans in order to complement a propagation incompetent virus and lead to the production of infectious viral particles. As used herein, the phrase “propagation competent” refers to a VSV virus that can be packaged into infectious viral particles without the need for G protein to be provided in trans. Accordingly, a propagation competent virus would not be restricted to a single round of viral particle production and would be able to be spread beyond primary infected cells.

The terms “protein”, “polypeptide” and “peptide” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include modifications, such as deletions, additions and substitutions (generally conservative in nature, but which may be non-conservative), to a native sequence, preferably such that the protein maintains the ability to elicit an immunological response within an animal to which the protein is administered. Also included are post-expression modifications, eg. glycosylation, acetylation, phosphorylation and the like.

The term “self molecule” refers to a molecule or antigen that may be a protein, polypeptide, fragment or fusion thereof that is produced by a host organism. A self molecule is a molecule or portion thereof which represents those produced by a host.

All patents, patent applications, and other literature cited herein are hereby incorporated by reference in their entirety.

The present invention is further illustrated and supported by the following examples. However, these examples should in no way be considered to further limit the scope of the invention. To the contrary, one having ordinary skill in the art would readily understand that there are other embodiments, modifications, and equivalents of the present invention without departing from the spirit of the present invention and/or the scope of the appended claims.

### EXAMPLES

### Example 1

### Generation, Amplification and Titration of rMuVgag and rVSVN4CT1gag1

rMuVgag1 Construct:

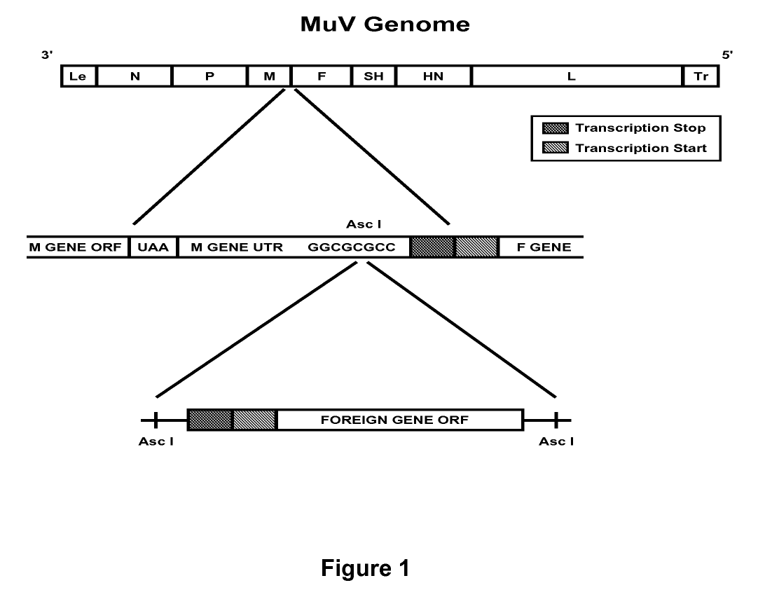

The construction and rescue of rMuV from genomic DNA has previously been described in detail (Clarke et al., 2000 J. Virol., 74: 4831-8) and published international patent application WO 2001/009309. The rMuV genomic cDNA was modified to enable insertion of a transcription unit(s) (TU) between the M and F genes for expression of a foreign gene(s). This was accomplished by generating two PCR products; one stretching from the unique BssHII site in the M gene into the M gene 3′ NCR and containing a primer encoded Ascl site at the 3′ end, and the other stretching from the M gene 3′ NCR to the unique Xhol site in the L gene and containing a primer encoded Ascl site at the 5′ end. Both PCR products were gel purified, digested with Ascl and then ligated in vitro. The resulting DNA fragment was gel purified, trimmed with BssHII and Xhol and cloned back into the rMuV genome cDNA. The HIV-1 HX-B2 p55 gag gene (SEQ ID NO.1) was then PCR amplified from an existing cDNA template (Egan et al., Vaccine 24: 4510-23), with primers encoding Ascl sites and MuV specific transcription stop/start signals (FIG. 1). The resulting PCR product was gel purified, trimmed with Ascl and cloned into the Ascl-containing rMuV genome cDNA. A helper-virus-free method for the recovery of rMuV from genomic cDNA has been described recently (Witko et al. 2006 J. Virol. Methods, 135:91-101) and was used for the rescue of rMuVgag. Briefly, near-confluent Vero cell monolayers in T-150 flasks were trypsinized, rinsed, collected by centrifugation at 300×g, resuspended in Iscove's Modified Dulbecco's Medium supplemented with 200 μM 2-mercaptoethanol, 1% nonessential amino acids, 1% sodium pyruvate and 1% DMSO, and then electroporated with a mixture of plasmids expressing T7 RNA polymerase under control of the human cytomegalovirus (CMV) promoter, and MuV N, P, and L proteins and a positive sense copy of the rMuVgag genome all under control of the T7 RNA polymerase promoter. Electroporated cells were collected by centrifugation at 300×g for 5 minutes at room temperature, and then transferred to a flask containing DMEM supplemented with 10% FBS, 200 μM 2-mercaptoethanol, 1% nonessential amino acids and 1% sodium pyruvate. Cells were then incubated at 37° C., 5% CO₂ for 3 hours, followed by heat shock at 43° C., 5% CO₂ for 3 hours and were then returned to 37° C., 5% CO₂ for approximately 8 hours, after which the medium was replaced and incubation was continued for 5-7 days. The cell monolayers were then scraped into suspension, agitated to break up cell clumps and transferred onto 50% confluent Vero cell monolayers in T-150 flasks. The cell monolayers were observed daily for the development of MuV induced cytopathic effect (CPE).

Rescued rMuVgag was plaque purified and amplified on Vero cell monolayers and then tested for gag expression by Western Blot, gag-specific ELISA and whole infected-cell immunofluorescence. The integrity of the gag open reading frame (ORF) in rMuVgag was also verified by consensus nucleotide sequencing of RT/PCR amplified cDNA spanning the gag TU. Working stocks of rMuV and rMuVgag were prepared by adsorption of virus to newly confluent Vero cell monolayers at a multiplicity of infection (moi) of 0.01 pfu/cell, followed by incubation at 37° C. for 48 hours in complete medium [DMEM containing 3.4 g/l sucrose and glutamine, supplemented with 10% FBS, and 1% sodium pyruvate]. Infected cell monolayers were then scraped into suspension and subjected to a single round of freeze-thaw in ethanol/dry ice followed by incubation in a 37° C. water bath. Cell debris was removed by centrifugation at 500×g, 4° C. for 10 minutes. The supernatant was flash frozen in an ethanol/dry ice bath and stored at −80° C. For titration of rMuV and rMuVgag, newly confluent Vero cell monolayers in six-well dishes were infected in duplicate with 10-fold dilutions of virus stock. Virus was adsorbed for 2 hours at 37° C. The inoculum was then replaced with 3 ml of complete medium containing ˜0.6-0.8% WN final concentration of molten agarose and cells were incubated at 37° C. for 4-5 days, after which the agarose overlay was removed and cell monolayers were fixed with 10% V/V formaldehyde in PBS for 10 minutes at room temperature. The formaldehyde solution was then replaced with PBS and virus plaques were counted under low power magnification on an inverted microscope.

rVSVN4CT1gag1 Construct:

The construction, rescue, in vitro and in vivo characterization of rVSVN4CT1gag1 has been previously described (Clarke et al. 2007 J. Virol., 81:2056-64, Roberts et al. 1999 J. Virol., 73: 3723-3732, Schnell et. al. 1998 EMBO J., 17: 1289-1296). The N gene was first deleted from rVSV_{IN} (Indiana strain) genomic cDNA by replacing the natural BsaA I/Xba I genome fragment with a DNA fragment that was generated by in vitro ligation of two PCR products; one stretching from the BsaA I site in the plasmid vector to the exact 3′ end of the virus Leader (Le) sequence (+sense), and the other spanning the transcription start signal of the P gene to the downstream Xba I site. Precise ligation of DNA containing the virus Le, with DNA containing the exact 3′ end of the P gene was achieved by addition of BsmB I sites to PCR primers. The N gene was then inserted back into the ΔN genome cDNA between the P and M genes (N2), between the M and G genes (N3), and between the G and L genes (N4) using a similar approach. For generation of the N2 genome cDNA, a PCR product spanning the entire N gene and 3° CT intergenic dinucleotide was ligated to flanking PCR fragments in vitro; one DNA fragment stretched from the unique Xba I site to the 3′ end of the P gene, and contained the P/M intergenic dinucleotide GT. A second DNA fragment spanned the entire M gene to the unique Mlu I site in the G gene. Addition of BsmB I sites to the 3′ and 5′ ends of the P, and M gene fragments respectively and to 3′ and 5′ ends of the N gene fragment, allowed all three DNA fragments to be ligated in vitro and then cloned into the Xba I and Mlu I sites of the ΔN genome cDNA. The N3 cDNA genome was constructed in a similar fashion. A PCR fragment spanning the unique Xba I site in the P gene to the end of the M gene including the 3° CT intergenic dinucleotide, was ligated to a PCR fragment spanning the entire N gene, a 3° CT intergenic dinucleotide and the first 32 nucleotides of the G gene containing the unique Mlu I site. Both DNAs were ligated through BsmB I sites at the 3′ end of the P/M fragment and 5′end of the N gene fragment. This DNA fragment was then cloned into the unique Xba I and Mlu I sites of the ΔN cDNA genome. For generation of the N4 genome cDNA, a PCR product spanning the entire N gene was joined with flanking PCR products; one stretching from the unique Mlu I site to the end of the G gene, including the 3′ CT intergenic di-nucleotide, and the other containing the G/L intergenic di-nucleotide CT, and the 5′ end of the L gene to the unique Hpa I site. All three fragments were joined by the addition of BsmB I sites to the 3′ and 5′ ends of the G and L gene fragments respectively, and to 3′ and 5′ ends of the N gene DNA fragment. The resulting contiguous DNA fragment was then cloned into the Mlu I and Hpa I sites of the ΔN cDNA genome.

The M_{NCP} gag5 and M_{NCP}CT1 gag5 vectors were generated by cloning a DNA fragment that spanned the mutant M_{NCP} gene and part of the P gene, into the unique Xba I/Mlu I sites of rVSV_{IN} cDNAs, containing either the HIV-1 Gag gene inserted between the G and L genes (rVSV_{IN} gag5) or the HIV-1 Gag gene inserted between a truncated (CT1) form of the G gene and the L gene (rVSV_{IN}CT1 gag5). For the purposes of this study, the rVSV_{IN}N3CT1 vector was modified to express HIV-1 p55 Gag protein from a transcription unit (TU) inserted in the first position of the genome, adjacent to the viral 3′ transcription promoter. To carry out this modification, three PCR products were generated: 1) a DNA fragment extending from the unique BsaA I site in the plasmid vector to the P gene 5′ untranslated region (UTR) of rVSV_{IN}N3CT1, including the P gene transcription start signal followed by eight additional non-coding nucleotides and a flanking Xho I site; 2) a fragment spanning from the P gene transcriptional start signal to the unique Xba I site in the P gene, including an Nhe I restriction site followed by a transcription termination signal, followed by a CT intergenic di-nucleotide, all added upstream of the P gene transcriptional start signal; and 3) a. DNA fragment contained the HIV-1 HXB2 strain p55 gag gene open reading frame (ORF) flanked by Xho I and Nhe I sites. The three DNA fragments were digested with Xhol and Nhe I and then ligated in vitro. The resulting ligation product was gel purified, BsaA I/Xba I digested and cloned into the BsaA I/Xba I sites of rVSV_{IN}N3CT1, to generate rVSV_{IN}N4CT1-gag 1.

Four N gene shuffle/CT combination mutants were generated by swapping the G genes from the N2 and N3 cDNAs with the CT1 and CT9 truncated forms of the G gene, via unique flanking Mlu I and Hpa I sites.

Recovery of rVSV from cDNA:

Infectious virus was recovered from genomic cDNA following transfection of BHK cells with a mixture of plasmids expressing VSV N, P and L proteins and full length positive sense genomic RNA, all under control of the bacteriophage T7 RNA polymerase transcription promoter (Lawson N.D. et al. 1995 Proc. Natl. Acad. Sci. USA, 92:4477-4481). For transfection, 95-100% confluent BHK cell monolayers in 6-well dishes were incubated at 32° C., 3% CO₂, for 4 hours in 4.5 ml/well of fresh growth medium. Meanwhile, a plasmid DNA/CaPO₄ precipitate was prepared for each cell monolayer by mixing 2-4 μg of plasmid containing the full length genomic cDNA, 1.0 μg of N plasmid, 0.5 μg of P plasmid, 0.15 μg of L plasmid, 25 μl of CaCl₂ (2.5M) and water to 250 μl final volume. The DNA/CaPO₄ precipitate was then formed by drop-wise addition of 250 μl of 2×BBS (280 mM NaCl, 50 mM BES, 1.5 mM Na₂HPO₄, pH 6.95-6.98) with gentle vortexing. The mixture was incubated at RT for 20 minutes to allow precipitate formation, and then added drop-wise to cells with gentle swirling. To provide a source of T7 RNA polymerase, MVA-T7-GK16 (Kovacs G. R. 2003 J. Virol. Methods, 111:29-36) was then added to each well at a moi of 3-4 pfu/cell, along with 20 μg/mi cytosine arabinoside (Ara-C) to inhibit amplification of MVA-T7. Cells were then incubated at 32° C., 3% CO₂ for 3 hours, followed by a 2 hour heat shock at 43° C., 3% CO₂ (Parks C. L. 1999 J. Virol., 3560-3566). Following heat shock, cells were incubated at 32° C., 3% CO₂ for 18-24 hours. Transfection medium was then replaced with 2 ml of fresh growth medium containing Ara C and cells were further incubated at 37° C., 5% CO₂ for 48-72 hours. Transfected cells were then scraped into suspension, gently pipetted repeatedly to reduce cell clumping and transferred to 95-100% confluent Vero cell monolayers in 6-well dishes. The following day, co-cultures were supplemented with 1 ml of fresh growth medium and incubation was continued for a further 3-5 days during which time VSV cytopathic effect (cpe) became apparent. Rescued virus was then triple plaque purified, and further amplified prior to in vitro and in vivo analysis.

### In Vitro Growth Studies:

For comparison of rVSV_{IN} mutant plaque size, plaque assays were performed in duplicate on replicate Vero cell monolayers as previously described. For growth kinetics studies, replicate Vero cell monolayers in 25 cm² flasks were infected in duplicate at a moi of 5 pfu/cell. Virus was adsorbed in 0.5 ml of growth medium for 15 minutes at RT, followed by 30 minutes at 37° C. with occasional rocking to prevent cell desiccation. After removal of the inoculum, monolayers were rinsed three times with 5mi of PBS to remove unbound virus; 5 ml of growth medium was then added to each monolayer and a 0.5 ml aliquot was immediately removed as ‘time zero’ (T₀) sample and replaced with 0.5mi of fresh medium. Incubation was continued at 37° C., 5% CO₂ for 48-72 hours, and further samples were taken at T₃-T₄₈. All samples were flash frozen in ethanol/dry ice and stored at −80° C. for titration.

Working stocks of rVSVN4CT1gag1 were prepared by adsorption of virus to confluent BHK cell monolayers at a multiplicity of infection (MOI) of 0.05 pfu/cell, followed by incubation at 32° C. for 48-60 hours. Cell debris was then removed from culture medium by centrifugation at 500×g, 4° C. for 10 minutes, and virus was purified by centrifugation (28,000 rpm, 4° C., for 90 minutes in a Beckman SW-28 rotor) through a sucrose cushion (10% wt/vol in PBS pH 7.0). Virus pellets were resuspended in PBS pH 7.0, flash frozen in an ethanol/dry ice bath and stored at −80° C.

### Western Blot Analysis of HIVgag Expression:

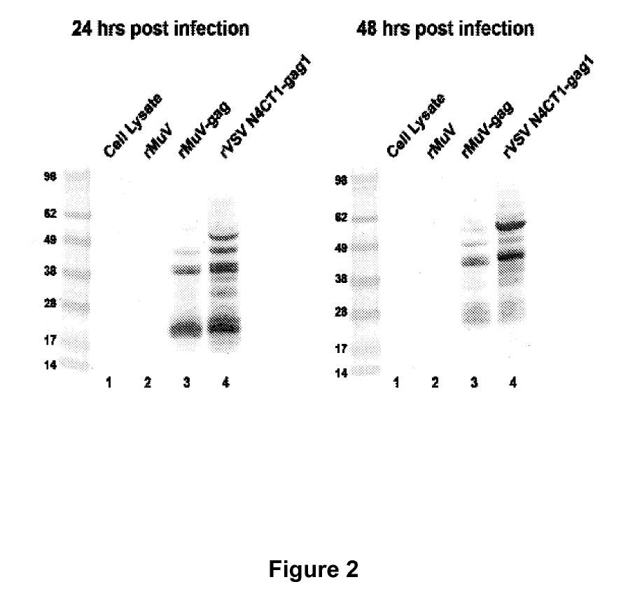

Replicate confluent BHK cell monolayers in six-well plates were infected at an MOI of 5 plaque-forming units per cell (PFU/ceII). Virus inoculum was adsorbed for 15 minutes at room temperature followed by 30 minutes at 37° C., 5% CO₂ for rVSVN4CT1gag1, and 2 hours at 37° C., 5% C02 for rMuVgag. Additional growth medium was then added and cells were incubated at 37° C., 5% CO₂ for 24-48 hours. At 24 hours post infection (HPI), cells were scraped into suspension and collected by centrifugation for 10 minutes at 3,000×g. Supernatant was removed and cell pellets were treated with 0.5 mL of lysis buffer (0.05 M Tris-HCL pH 7.5, 0.01 M NaCl, 1X Triton). Cell lysates were then diluted 1:1 in Laemmli Sample Buffer and heated at 90° C. for 5 minutes to denature proteins. Samples were electrophoresed on 4-12% Bio-Tris-PAGE gels (NuPAGE Cat. # NP0321) with a Precision Plus Protein Standard (Bio Rad. Cat. #1610375), and proteins were then transferred to nitrocellulose membrane using the iBlot system (Invitrogen Cat. #IB1001, IB3010-02). The nitrocellulose membrane was then blocked in 5% milk in TTBS (0.02% Tween 20; 0.9% NaCl; 100 mM Tris-HCl, pH 7.5) overnight, followed by three 5-minute washes in TTBS. The blot was incubated with HIV-1 p24 Gag specific monoclonal antibody (ImmunoDiagnostics, Inc. Cat. #1103), diluted 1:2000 in 5% milk/TTBS, for 1 hour at room temperature followed by three five-minute washes in TTBS. The blot was then incubated with secondary antibody, biotinylated goat anti-mouse IgG (Vector Labs, Cat.# BA-9200), diluted 1:2000 in 5% milk/TTBS, for 1 hour followed by three 5-minute washes in TTBS. Protein-antibody complexes were then visualized using Vectastain ABC and TMB substrate kits (Vector Labs, Cat.# PK-6100, SK4400). As seen in FIG. 2, a robust expression of gag protein was detected by Western blot analysis of rMuVgag using both the rMuVgag (lane 3) and rVSVN4CT1gag1 (lane 4) infected cells. Empty rMuV (lane 2) showed no gag expression.

### Example 2

### Immunization of Non-human Primates with rMuVgag and rVSVN4CT1gag1

A total of 15, captive-bred, rhesus macaques (Macaca mulatta) of Indian origin were used in this study. Macaques were maintained in accordance with the Guide for the Care and Use of Laboratory Animals (National Research Council, National Academic Press, Washington, D.C., 1996). All animals were seronegative for MuV, VSV, and HIV-1 gag prior to the start of the study. Two groups of 5 animals were inoculated subcutaneously (SC) at two dorsal sites with 1×10⁷ pfu/animal of rMuVgag contained in 2 ml total volume (1 ml/site). A third group was inoculated IM in each quadricep with 5×10⁷ pfu/animal of rVSVN4CT1gag1 contained in 2 ml total volume (1 ml/site) (Table 1). Eight weeks later, animal groups primed with rMuVgag were boosted with either a second SC dose of rMuVgag (1×10⁷ pfu/animal) or an IM dose of rVSVN4CT1gag1 (5×10⁷ pfu/animal), and animals primed with rVSVN4CT1gag1 were boosted with a SC dose of rMuVgag (1×10⁷ pfu/animal). Animals receiving rMuVgag twice were then also boosted with rVSVN4CT1gag1 (5×10⁷ pfu/animal) 8 weeks after the second rMuVgag inoculation. Blood samples collected from animals at intervals pre- and post inoculation and were processed for measurement of humoral and cellular immune responses specific for HIV-1 gag, rVSV and rMuV proteins.

TABLE 1

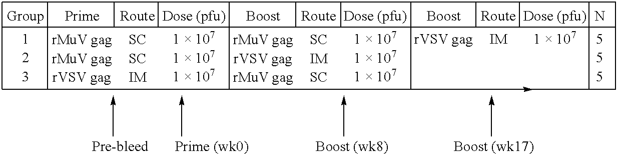

### Example 3

### Quantitation of gag-Specific Immune Responses of Non-human Primates Immunized in Example 2

### Analysis of Interleukin-2 (IL-2) and Interferon Production:

The filter immunoplaque assay, otherwise called the enzyme-linked immunospot assay (ELISpot), was initially developed to detect and quantitate individual antibody-secreting B cells. The technique originally provided a rapid and versatile alternative to conventional plaque-forming cell assays. Recent modifications have improved the sensitivity of the ELISpot assay such that cells producing as few as 100 molecules of specific protein per second can be detected. These assays take advantage of the relatively high concentration of a given protein (such as a cytokine) in the environment immediately surrounding the protein-secreting cell. These cell products are captured and detected using high-affinity antibodies.

The ELISpot assay utilizes two high-affinity cytokine-specific antibodies directed against different epitopes on the same cytokine molecule: either two monoclonal antibodies or a combination of one monoclonal antibody and one polyvalent antiserum. ELISpot generates spots based on a colorimetric reaction that detects the cytokine secreted by a single cell. The spot represents a “footprint” of the original cytokine-producing cell. Spots (i.e., spot forming cells or SFC) are permanent and can be quantitated visually, microscopically, or electronically.

Ninety-six-well flat-bottom ELISpot plates (Millipore, Bedford, Mass.) were coated overnight with a mouse anti-human (-interferon (hIFN-γ) monoclonal antibody (clone 27, BD-Pharmingen, San Diego Calif.) at a concentration of 1 μg/mL, or with a goat anti-human IL-2 polyclonal antibodies (R&D system, Minneapolis, Minn.) at manufacturers recommended concentration. Thereafter, the plates were washed three times with 1× phosphate buffered saline (1×PBS) and then blocked for 2 hours with PBS containing 5% heat inactivated (HI) fetal bovine serum (FBS). EDTA anti-coagulated rhesus macaque whole blood was collected at various time points after immunization, peripheral blood lymphocytes (PBLs) were isolated from whole blood by Ficoll-Hypaque density gradient centrifugation and resuspended in complete R05 culture medium (RPMI 1640 medium supplemented with 5% FBS, 2 mM L-glutamine, 100 units/mL penicillin, 100 μg/mL streptomycin sulfate, 1 mM sodium pyruvate, 1 mM HEPES, 100 mM non-essential amino acids) and shipped via overnight courier. Within 24 hours of blood draw, the isolated macaque PBLs were washed once with complete R05 culture medium and resuspended in complete R05 culture medium containing either 50 μg/mL PHA-M (Sigma), peptide pools (15mers overlapping by 11 amino acids; 1 mcM each final peptide concentration) spanning HIV_{HXB2} gag p55 or medium alone. Input cell numbers were 2×10⁵ PBLs per well (2×10⁶ PBLs/mL), and assayed in duplicate wells. Cells were incubated for 18-24 hours at 37° C. and then removed from the ELISpot plate by first washing with de-ionized water and then washing six times with 1×PBS containing 0.25% Tween-20 and three additional times with 1×PBS. Thereafter, IFN-γ ELISPOT plates were treated with a rabbit polyclonal anti-human IFN-γ biotinylated detection antibody (0.2 μg/well, Biosource, Camarillo, Calif.) diluted with 1×PBS containing 1% bovine serum albumin (BSA) and incubated at room temperature for two hours; IL-2 ELISPOT plates were treated with goat anti-human IL-2 biotinylated detection antibodies (R&D system, Minneapolis, Minn.) at manufacturers recommended concentration diluted with 1×PBS containing 1% BSA and incubated at 4° C. for overnight. ELISpot plates were then washed 10 times with 1×PBS containing 0.25% Tween-20 and treated with 100 μL per well of streptavidin-horseradish peroxidase conjugate (BD-Biosciences, San Diego Calif.) diluted 1:500 (for IFN-γ) or 1:100 (for IL-2) with 1×PBS containing 5% FBS and 0.005% Tween-20 and incubated an additional one hour at room temperature. Unbound conjugate was removed by rinsing the plate ten times with 1×PBS containing 0.25% Tween-20. Chromogenic substrate (100 mUwell, 1-step NBT/BCIP, Pierce, Rockford, Ill.) was then added for 3-5 minutes (for IFN-γ) or 25-30 minutes (for IL-2) before being rinsed away with water, after which the plates were air-dried, and the resulting spots counted using an Immunospot Reader (CTL Inc., Cleveland, Ohio). Peptide-specific IFN-γ or IL-2 ELISpot responses were considered positive if the response (minus media background) was >3 fold above the media response and ≧50 SFC/10⁶ PBLs.

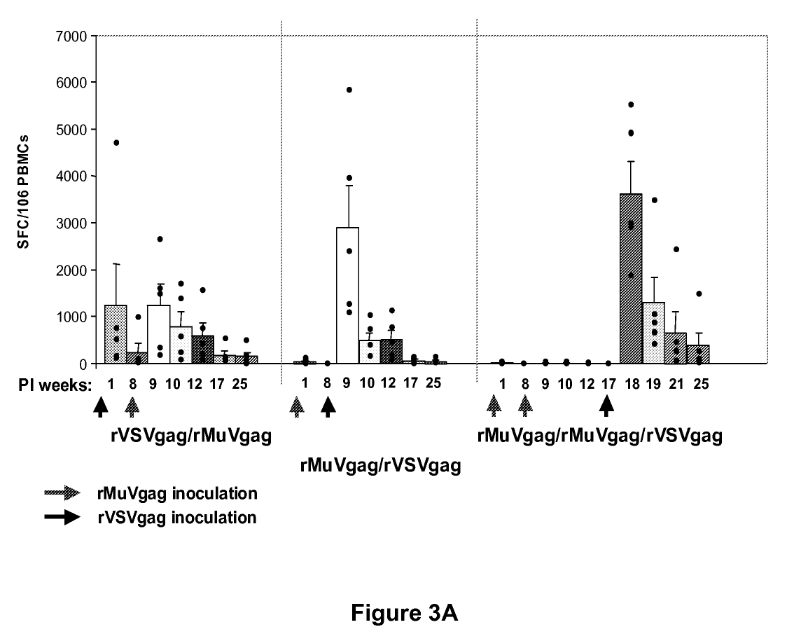

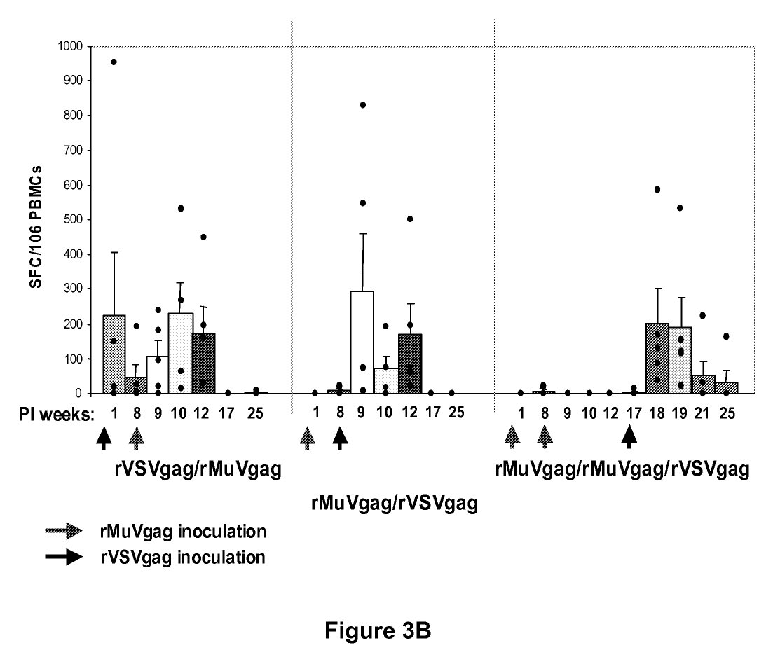

Animals receiving rVSVN4CT1gag1 had a robust T-cell response (average approximately 1000 IFN-γ SFC/10⁶ peripheral blood mononuclear cells (PBMCs) by week one post prime (FIG. 3A). By week 8 post prime, responses in these animals had declined to an average of approximately 200 IFN-γ ELISPOTS, and were boosted back to an average of approximately 1000 IFN-γ ELISPOTS one week after immunization with rMuVgag. Again, these responses waned to approximately 150 IFN-γ ELISPOTS by week 17 post boost (week 25 post prime). The pattern of IL-2 ELISPOT responses measured in these animals was very similar to that obtained for IFN-γ ELISPOTS (FIG. 3B), but at a reduced magnitude. Again, IL-2 ELISPOT responses peaked at approximately 200 SFC/10⁶ PBMCs one week post prime with rVSVN4CT1gag1, declining to approximately 50 ELISPOTS by week 8 post prime, and boosted back to approximately 200 ELISPOTS two weeks post inoculation with rMuVgag. By week 17 post-boost with rMuVgag, IL-2 ELISPOT responses were undetectable.

In contrast to the robust gag-specific responses detected in animals primed with rVSVN4CT1gag1, gag-specific IFN-γ and IL-2 ELISPOT responses detected in animals primed either once or twice with rMuVgag were virtually undetectable at week one post inoculation. However, robust peak of IFN-γ ELISPOT responses (3000-3500 SFC/10⁶ PBMCs) were detected in animals primed either once or twice with rMuVgag and then boosted with rVSVN4CT1gag1. Also notable, in all 10 animals primed with either one or two doses of rMuVgag and boosted with rVSVN4CT1gag1, peak responses of >1000 gag-specific IFN-γ ELISPOTs were detected. Although these responses waned quickly, approximately 200 IFN-γ ELISPOTs could still be detected 8 weeks after boosting with rVSVN4CT1gag1, in animals receiving two rMuVgag priming doses. The pattern of IL-2 ELISPOT responses in these animals, mirrored IFN-γ ELISPOT responses, but at reduced levels. Also, differences in the magnitude of peak IL-2 ELISPOT responses detected for all three different immunization regimens was not as remarkable as those seen for IFN-γ ELISPOT responses.

### In Vitro Stimulation and Staining of Cells:

Fresh isolated PBMCs were resuspended at approximately 1×10⁷ cells/ml in R05 culture medium and stimulated with 1 μM gag peptide mix for 5-6 hours at 37° C. in the presence of brefeldin A (GolgiPlug, 1 μL/ml; BD Biosciences). Negative control tubes without peptide were also included. After stimulation, cells were washed twice in FACS washing buffer (PBS/2% FBS) and stained for 20 minutes in the dark at 4° C. with surface marker monoclonal antibodies: anti-CD3-Pacific Blue, anti-CD4-PerCP-Cy5.5, anti-CD8-APC-Cy7. Aqua was used to stain the dead cells. Cells were washed and permeablized according to the manufacturer's instructions (Cytofix/Cytoperm kit, BD Biosciences). After washing twice in the supplied buffer, cells were intracellularly stained with antibodies for cytokines: anti-IFN-δ Alex 700, anti-TNF-α-PE-Cy7 and anti-IL2-FITC at ice for 30 minutes. Cells were subsequently washed in the supplied buffer and fixed in BD™ stabilizing Fixztive buffer (BD Biosciences). Fixed cells were stored at 4° C. in the dark until cytometric analysis (performed within 24 hours).

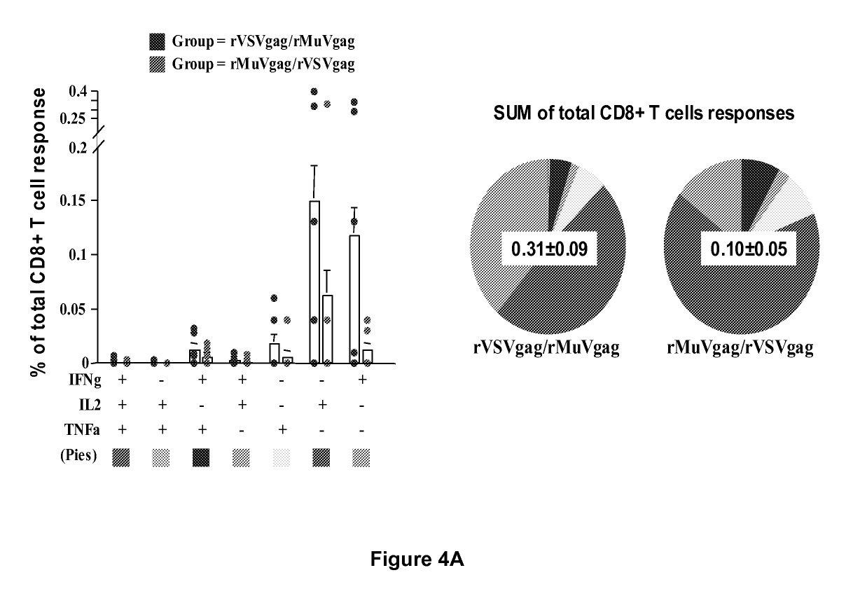

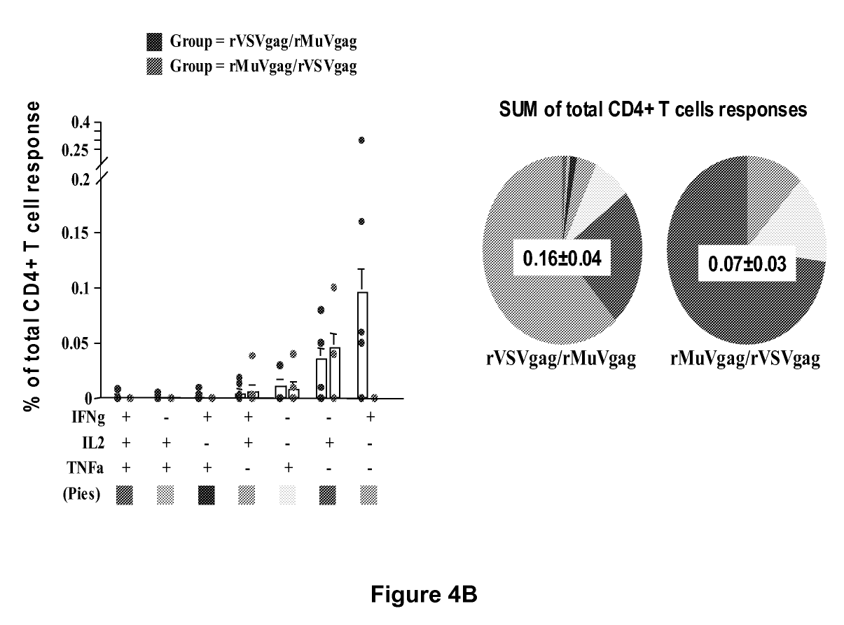

As seen in FIGS. 4A and 4B, respectively, intracellular cytokines were measured in gag-specific CD8⁺ and CD4⁺ by ICS 25 weeks after primary inoculation to assess differences in memory responses between rVSVN4CT1gag1/rMuVgag and rMuVgag/rVSVN4CTgag1 immunization regimens. ICS was not performed for animals primed twice with rMuVgag and boosted with rVSVN4CT1gag1. Overall the percentage of CD8⁺ and CD4⁺ T cells producing cytokines was low for both immunization regimens, and the number of cells producing either IFN-γ or IL-2 or TNF-α was greater than those producing two or more cytokines.

Detection of MuV specific IgG:

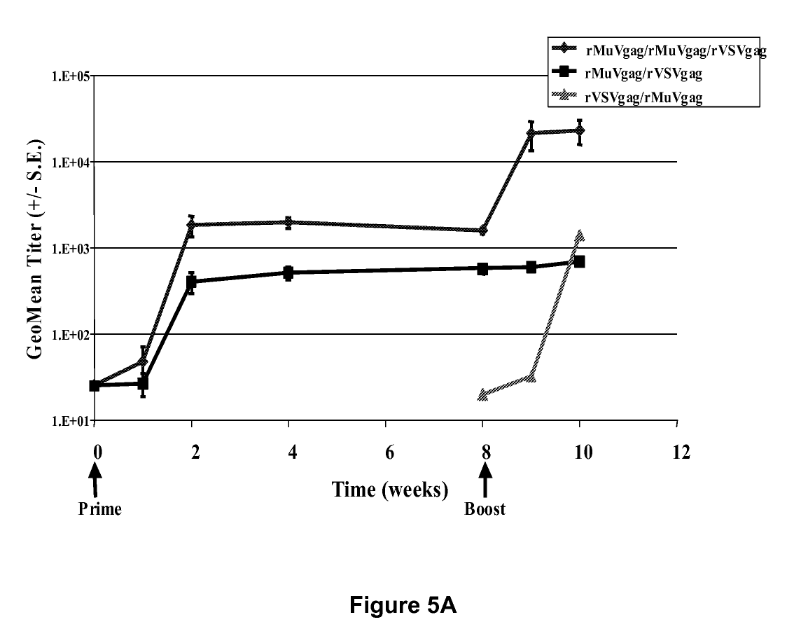

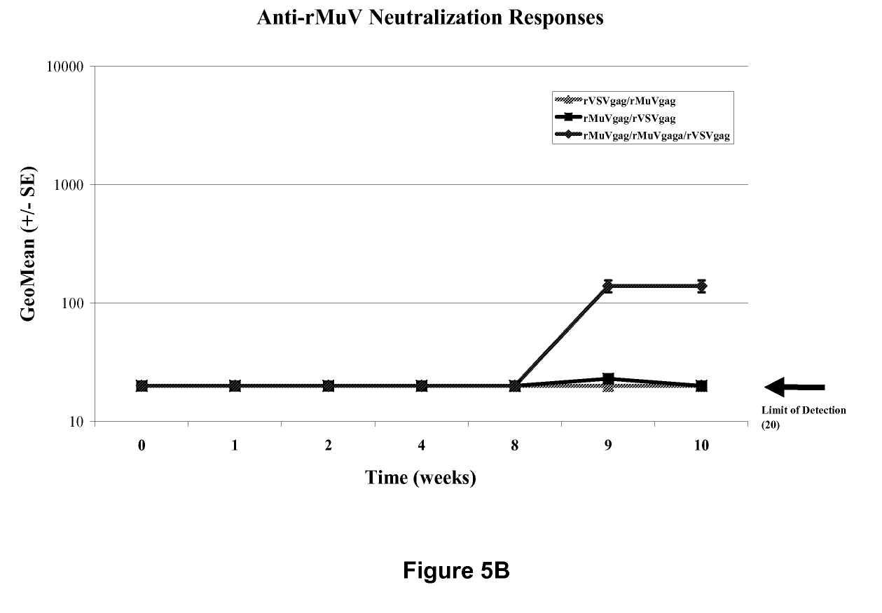

Animal sera were tested for the presence of MuV-specific IgG using an ELISA kit supplied by Diagnostic Automation Inc. Briefly, serial two-fold dilutions of serum samples were prepared in the provided serum diluent, and then added to MuV-antigen coated 96 well plates. Plates were incubated for 30 minutes at room temperature (RT) and then rinsed to remove serum samples. Horseradish-peroxidase (HRP) conjugated rabbit α-IgG was then added to each well for 30 minutes at RT. The HRP conjugate was then rinsed off, and TMB substrate solution was added to allow color development, which was stopped by addition of 1N H₂SO₄. Colorimetric analysis was performed at 450 nm using a Biotek plate reader (Biotek, Winooski, Vt.). The Log₁₀ of serum dilutions were plotted against absorbance values and the data was transformed into a linear regression using Origin 6.1 software. The geometric mean titers plus the standard error were determined for each group. As seen in FIG. 5A inoculation of animals with a single dose of rMuVgag elicited a measurable MuV-specific IgG response, which was significantly boosted following a second rMuVgag inoculation.

### MuV Neutralization Assay:

Neutralizing antibodies were determined by standard virus neutralization assay. Briefly, 100-200 pfu of rMuV was incubated with duplicate two-fold dilutions of serum for 1 hour at 37° C. and then added to freshly confluent Vero cell monolayers in 96 well dishes. Cells were then incubated at 37° C., 5% CO₂ for 4 days, and monitored for viral cpe under the microscope. The neutralization titer was calculated as the reciprocal of the highest serum dilution that completely protected cell monolayers from viral cpe. As can be seen in FIG. 5B, similar results were obtained in MuV neutralization assays as was seem in the MuV ELISA where neutralization titers were very low following a single dose of rMuVgag, but increased significantly after a second rMuVgag inoculation.

### Example 4

### rMuVgag/rVSVN4CT1gag1 Heterologous Prime-Boost Immunization of Rhesus Macaques that had Pre-existing Immunity to Mumps Virus

The Examples above have shown that priming MuV naive rhesus macaques once or twice with rMuVgag followed by a boost with rVSVN4CT1gag1 elicited very robust HIV-1 gag-specific ELISPOT responses of 3,000-3,500/10⁶ in peripheral blood lymphocytes (PBLs). To further examine the potential utility of the rMuVgag vector for similar prime-boost studies in humans, where there is a known and measurable pre-existing immune response to MuV due to natural infection and childhood vaccination programs, a simple prime-boost vaccination regimen was performed in macaques that were pre-immunized twice with rMuV (‘empty’ mumps virus).

Five macaques that were immunologically naive for MuV, VSV and HIV-1 were chosen for the study. These animals were immunized twice with 10⁷ PFU of rMUV (‘empty’), with an 8 wk interval between immunizations. Following each immunization, MuV-specific IgG and MuV-specific neutralization activity in animal sera were monitored by ELISA and a standard neutralization assay, respectively. When average serum neutralizing activity (and IgG level) in immunized macaques was at a level similar to that observed in a random sample of 100 humans, aged 18-30 yrs, the macaques were primed with 10⁷ PFU of rMuVgag and then boosted 8 wks later with 5×10⁷ PFU of rVSVN4CT1gag1. After prime and boost immunizations, HIV-1 gag-specific ELISPOT responses, and MuV and VSV-specific humoral responses were measured.

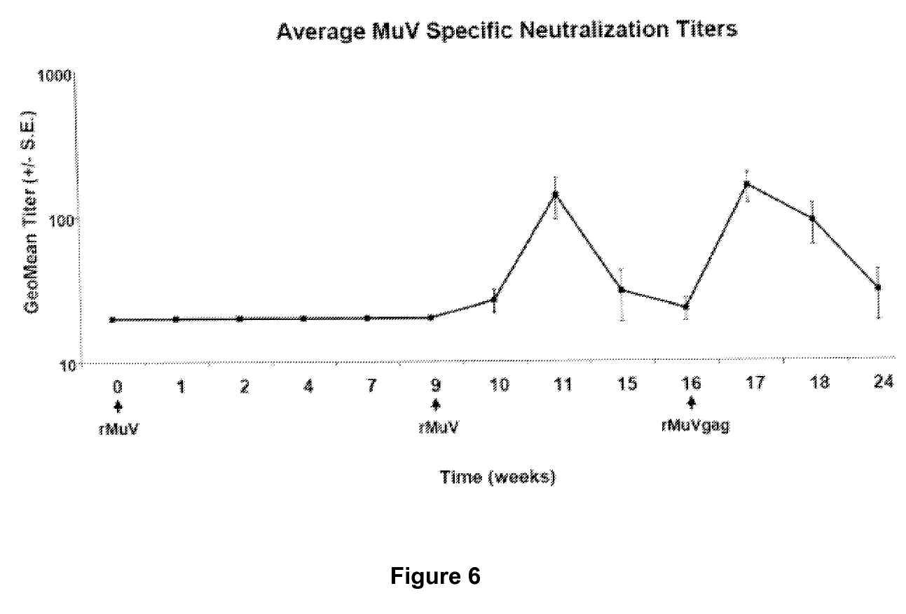

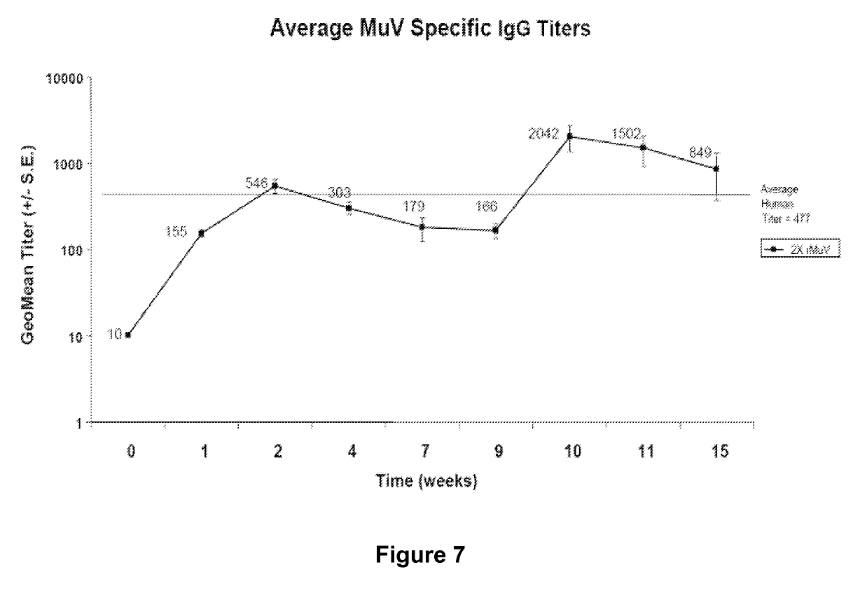

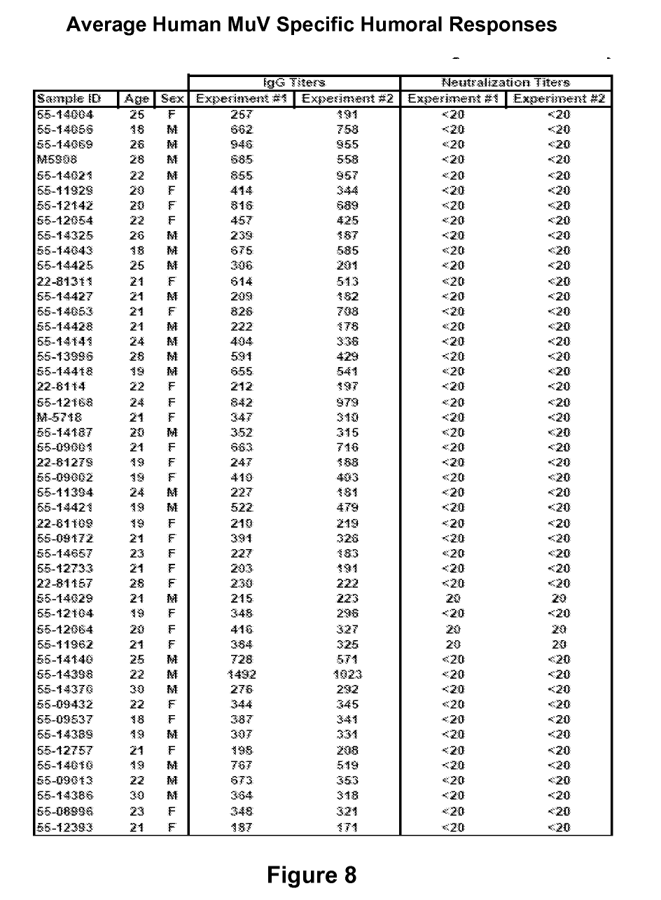

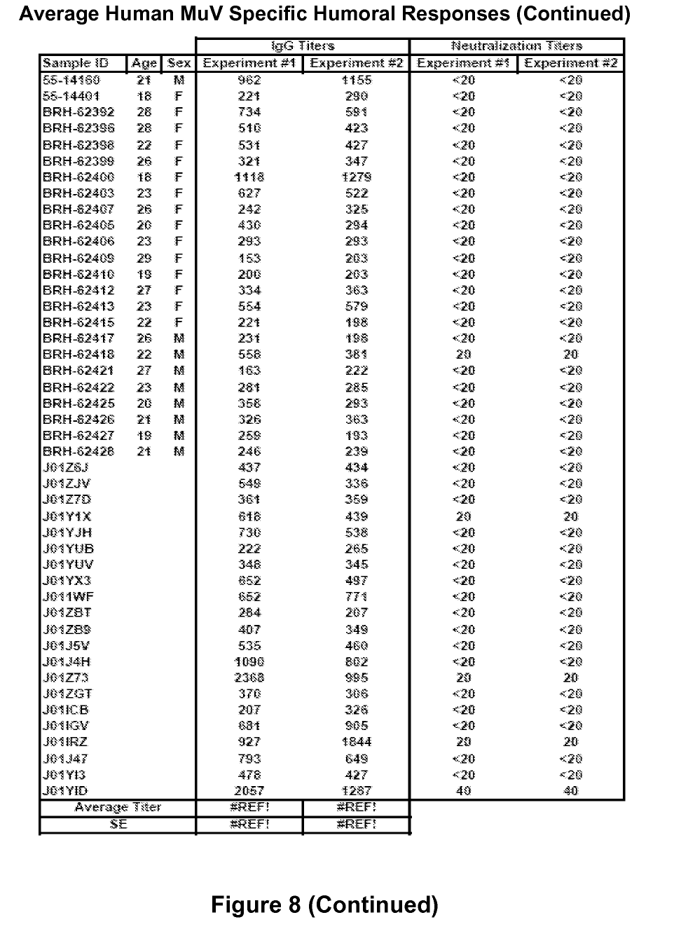

After a single inoculation of rMuV (‘empty’) very little MuV-specific neutralizing antibody was detected. However, after the second inoculation with rMuV (‘empty’), MuV-specific neutralizing activity rose, at least ten-fold, as shown in FIG. 6. The average peak neutralizing titer obtained after the second rMuV inoculation was higher than that observed for the average neutralizing titer present in 18-30 year old humans (FIG. 8). Neutralizing activity then declined quickly in macaques to a level that was similar to that seen in humans, but boosted rapidly to levels observed after the second pre-immunization following inoculation with rMuVgag. A similar pattern of MuV-specific IgG response was also observed in macaques following rMuV inoculation; accordingly macaques were inoculated with rMuVgag when MuV-specific IgG levels were equivalent to the average IgG level detected in human sera (FIGS. 7, and 8).

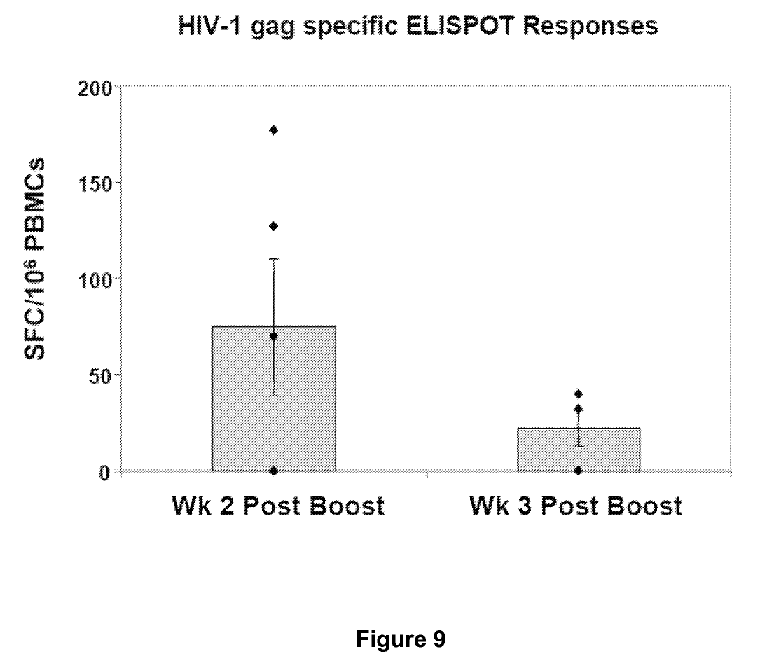

As seen in the previous example there was no detectable gag-specific ELISPOT response in macaques following inoculation with rMuVgag. After boosting with rVSVN4CT1gag1 there appeared to be little enhancement of gag-specific ELISPOT responses (FIG. 9) relative to previous studies where macaques were inoculated with only a single dose of rVSVN4CT1gag1, indicating that there was little or no gag-specific priming activity by rMuVgag in rMuV pre-immunized animals. In contrast, a very robust gag-specific ELISPOT responses was previously observed in MuV-naïve animals that were primed with rMuVgag and boosted with rVSVN4CT1gag1. The ELISPOT data set was complicated by the apparent failure of the peptide pool to stimulate proliferation of gag-specific PBLs at week one post boost with rVSVN4CT1gag1 (a time-point that would normally yield peak gag-specific ELISPOT responses following rVSVN4CT1gag1 inoculation). Consequently, a peptide pool from another laboratory was used for the week 2 post-boost stimulation of PBLs. Results from that assay indicated ˜150 ELISPOTS/10⁶ PBLs, consistent with levels previously observed at the same time interval, following inoculation of macaques with a single dose of rVSVN4CT1gag1 in the absence of any priming inoculations. Titration of retention samples from the study clearly indicated that macaques had received the anticipated doses of rMuVgag and rVSVN4CT1gag1, and a VSV neutralization assay demonstrated that macaques had sero-converted following inoculation with rVSVN4CT1gag1, indicating a successful ‘take’ of the vaccine.

This example demonstrates that the induction and presence of measurable MuV specific neutralizing activity in rhesus macaques prevents effective priming of a gag-specific T-cell response by rMuV gag and subsequent boosting with rVSVN4CT1gag1. It is believed that the prevention of significant replication and spread of rMuVgag in animals following inoculation with empty rMuV is the presence or rapid induction of neutralizing antibody, as indicated by the spike in neutralizing antibody detected one week after rMuVgag inoculations. However, this data does confirm that in order to fully sero-convert a naive macaque, two inoculations of rMuV is required. The second inoculation does, in fact, induce a large increase in humoral immunity, indicating a solid “take” of the second dose of virus.

### Example 5

### Alteration of Neutralizing Antibody Epitopes on MuV

In order to bypass the immune response that elicits neutralizing antibodies that prevent the replication and spread of rMuVgag after inoculation with MuV, the immunogenic domains within one or both of the surface glycoproteins, F and HN, on the mumps virus are altered. Effectively altering such domains effectively reduces the likelihood that an rMuV vaccine vector is recognized and neutralized by the pre-existing immunity resulting from previous exposure to MuV by inoculation or natural infection.

It has been shown that the HN protein, Genbank accession number AAL83745, (SEQ ID NO. 2) of MuV is a major target for a humoral immune response. The HN protein has four domains, HN 1-4, wherein HN3 (SEQ ID NO. 3), amino acids 213-372, has been shown to elicit neutralizing antibodies. Within HN3 particular epitopes have been determined, particularly (but not limited to) amino acids 352-360 (SEQ ID NO.4) (Cusi et al. Virus Research 74(2001) 133-137 and Kulkarni-Kale et al. Virology (2007), 436-446). Alteration of the epitopes within HN3 using the genomic cDNA encoding amino acids 352-360 of the HN protein of rMuVgag is performed using site directed mutagenesis and techniques well known to those skilled in the art. A helper-virus free method for the recovery of the altered rMuVgag is undertaken, as previously described. Further isolation of the altered rMuVgag and testing for expression of rMuVgag proteins is performed using the procedures as described in Example 1. To assess the ability of the altered rMuVgag to escape neutralization by MuV-specific antibodies, the altered rMuVgag is further tested in standard neutralization assays with serum collected from human subjects. Initially a prime-boost testing regimen using rhesus macaques having previous exposure to rMUV, as described in Example 4, are undertaken. A similar alteration of rMuV using mumps surface glycoprotein F, Genbank accession number ML83743, (SEQ ID. NO. 5) is performed as described above. Analogous studies are performed in human subjects.

What is claimed is:

1. A method for generating an antigen-specific immune response in a subject, the method comprising in sequential order the steps of:
(a) administering to the subject at least one dose of a first immunogenic composition comprising a recombinant mumps virus (rMuV) comprising a nucleic acid sequence encoding a heterologous antigen under the control of a regulatory sequence that directs expression of the antigen by the recombinant rMuV; followed by
(b) administering to the subject at least one dose of a second immunogenic composition comprising a recombinant vesicular stomatitis virus (rVSV) comprising a nucleic acid sequence encoding a heterologous antigen under the control of a regulatory sequence that directs expression of the antigen by the recombinant rVSV;
wherein the antigen expressed in step (a) may be the same as or different from the antigen expressed in step (b).

2. The method according to claim 1 wherein the first immunogenic composition further comprises an alteration in an rMuV protein.

3. The method according to claim 2 wherein the altered rMuV protein comprises at least one of an altered epitope in the HN protein and an altered epitope in the F protein.

4. The method of claim 3 wherein the alteration in the at least one of an altered epitope in the HN protein and an altered epitope in the F protein produces an rMuV that is not recognized by a pre-existing neutralizing antibody response to MuV in the subject.

5. The method according to claim 1 wherein at least one of said immunogenic compositions is administered in a pharmaceutically acceptable diluent.

6. The method according to claim 1 wherein the first immunogenic composition is administered to the subject at least two times prior to administering to the subject the second immunogenic composition.

7. The method according to claim 1 wherein the subject is a human.

8. The method according to claim 1 wherein at least one of the rMuV and rVSV is attenuated.

9. The method according to claim 1 wherein the rVSV is attenuated and expresses a G protein having a truncated cytoplasmic tail (CT) domain.

10. The method according to claim 9 wherein the attenuated rVSV expresses a G protein having the cytoplasmic tail domain truncated to one amino acid (CT1).

11. The method according to claim 9 wherein attenuated rVSV expresses a G protein having a cytoplasmic tail domain truncated to nine amino acids (CT9).

12. The method according to claim 1 wherein the rVSV is attenuated and comprises a translocation of the N gene to a different, distal position in the rVSV genome.

13. The method according to claim 1 wherein at least one of the rMuV and rVSV is propagation incompetent.

14. The method according to claim 13 wherein the rVSV is propagation incompetent and lacks a VSV G protein (VSVΔG).

15. The method according to claim 13 wherein the rVSV is propagation incompetent and expresses a G protein having a truncated extracellular domain (rVSV-Gstem).

16. The method according to claim 1 wherein the rMuV is attenuated and is derived from the Jeryl Lynn strain.

17. The method of claim 1 which further comprises administering to the subject one or more adjuvants.

18. The method according to claim 1 wherein the antigen is a protein, wherein the protein is derived from a source selected from the group consisting of a bacterium, virus, fungus, parasite, a cancer cell, a tumor cell, an allergen and a self-molecule.

19. The method according to claim 18 wherein the antigen is selected from the group consisting of: an HIV antigen, an HTLV antigen, an SIV antigen, an RSV antigen, a PIV antigen, an HSV antigen, a CMV antigen, an Epstein-Barr virus antigen, a Varicella-Zoster virus antigen, a mumps virus antigen, a measles virus antigen, an influenza virus antigen, a poliovirus antigen, a rhinovirus antigen, a hepatitis A virus antigen, a hepatitis B virus antigen, a hepatitis C virus antigen, a Norwalk virus antigen, a togavirus antigen, an alphavirus antigen, a rubella virus antigen, a rabies virus antigen, a Marburg virus antigen, an Ebola virus antigen, a papilloma virus antigen, a polyoma virus antigen, a metapneumovirus antigen, a coronavirus antigen, a Vibrio cholerae antigen, a Plasmodium falciparum antigen, a Plasmodium vivax antigen, a Plasmodium ovate antigen, a Plasmodium malariae antigen, a Plasmodium knowlesi antigen, a Streptococcus pneumoniae antigen, Streptococcus pyogenes antigen, a Helicobacter pylon antigen, a Streptococcus agalactiae antigen, a Neisseria meningitidis antigen, a Neisseria gonorrheae antigen, a Corynebacteria diphtheriae antigen, a Clostridium tetani antigen, a Bordetella pertussis antigen, a Haemophilus antigen, a Chlamydia antigen, a Escherichia coli antigen, a cytokine, a T-helper epitope, and a CTL epitope.

20. The method according to claim 19 wherein the antigen comprises an HIV antigen.

21. The method according to claim 20 wherein the HIV antigen comprises a protein encoded by a gene selected from the group consisting of gag, env, pol, vif, nef, tat, vpr, rev or vpu.

22. The method according to claim 21 wherein the HIV antigen is a gag protein.

23. The method according to claim 22 wherein the gene encoding the gag protein comprises SEQ ID NO.1.

24. The method according to claim 1 wherein the immune response comprises an increase in cell mediated immunity greater than that achieved by administering the first or second immunogenic compositions alone.

25. The method according to claim 1 wherein the immune response comprises an increase in antibody response to the antigen greater than that achieved by administering the first or second compositions alone.

26. The method according to claim 1 which further comprises administering to the subject a second antigen.

27. The method according to claim 1 wherein at least one of the first and second immunogenic compositions is administered by a route selected from the group consisting of: intravenous, intradermal, subcutaneous, intramuscular, intraperitoneal, oral, rectal, intranasal, buccal, and vaginal.

28. The method according to claim 27, wherein the first immunogenic composition is administered using an intramuscular or a subcutaneous route.

29. The method according to claim 27 wherein the second immunogenic composition is administered using an intramuscular or a subcutaneous route.

30. The method according to claim 1 wherein the second immunogenic composition is administered at least once between about 4 weeks and about 10 weeks after the first immunogenic composition is administered at least once.

31. The method according to claim 30 wherein the second immunogenic composition is administered at least once between about 7 weeks and about 9 weeks after the first immunogenic composition is administered at least once.

32. The method according to claim 30 wherein the second immunogenic composition is administered at least once at about 8 weeks after the first immunogenic composition is administered at least once.

33. An immunogenic composition for generating an antigen-specific immune response in a subject, the immunogenic composition comprising:
(a) a first immunogenic composition comprising a recombinant mumps virus (rMuV) comprising a nucleic acid sequence encoding a heterologous antigen under the control of a regulatory sequence that directs expression of the antigen by the recombinant rMuV; and
(b) a second immunogenic composition to be administered after the first immunogenic composition, said second composition comprising a recombinant vesicular stomatitis virus (rVSV) comprising a nucleic acid sequence encoding a heterologous antigen under the control of a regulatory sequence that directs expression of the antigen by the recombinant VSV;
wherein the antigen expressed in the first composition may be the same as or different from the antigen expressed in the second composition.

34 The immunogenic composition of claim 33 wherein the first immunogenic composition further comprises an alteration in an rMuV protein.

35. The immunogenic composition of claim 34 wherein the altered rMuV protein comprises at least one of an altered epitope in the HN protein and an altered epitope in the F protein.

36. The immunogenic composition of claim 35 wherein the alteration in at least one of an altered epitope in the HN protein and an altered epitope in the F protein produces an rMuV that is not recognized by a pre-existing neutralizing antibody response to MuV in the subject.

37. The immunogenic composition of claim 33 wherein the subject is a human.

38. A kit for generating an antigen specific response in a subject, the kit comprising a first immunogenic composition comprising a recombinant mumps virus (rMuV) comprising a nucleic acid sequence encoding a heterologous antigen under the control of a regulatory sequence that directs expression of the antigen by the recombinant rMuV, and a second immunogenic composition to be administered after the first immunogenic composition comprising a recombinant vesicular stomatitis virus (rVSV) comprising a nucleic acid sequence encoding a heterologous antigen under the control of a regulatory sequence that directs expression of the antigen by the recombinant rVSV, wherein the antigen expressed in the first composition may be the same as or different from the antigen expressed in the second composition.

39. The kit of claim 38 wherein the first immunogenic composition further comprises an alteration in an rMuV protein.

40. The kit of claim 39 wherein the altered rMuV protein comprises at least one of an altered epitope in the HN protein and an altered epitope in the F protein

41. The kit of claim 40 wherein the alteration in the at least one of an altered epitope in the HN protein and an altered epitope in the F protein produces an rMuV that is not recognized by a pre-existing neutralizing antibody response to MuV in the subject.

42. The kit of claim 38 wherein at least one of the rMuV and rVSV is propagation competent.

43. The kit of claim 38 wherein at least one of the rMuV and rVSV is propagation incompetent.

44. The kit of claim 38 further comprising an adjuvant.
